(19) United States
(12) Patent Application Publication
(10) Pub. No.: US 2008/0261215 A1
(43) Pub. Date: Oct. 23, 2008

(54) Method for Evaluating the Allergen Sensitivity of an Individual

(75) Inventors: Ashok Purohit, Strasbourg (FR); Carine Metz-Favre, Strasbourg (FR); Sylvia Laffer, Vienna (AT); Rudolf Valenta, Theresienfeld (AT); Nadine Mothes-Luksch, Vienna (AT); Peter Valent, Vienna (AT); Angele Verot, La Wantzenau (FR); Gabrielle Pauli, Strasbourg (FR)

Correspondence Address: CHALKER FLORES, LLP, 2711 LBJ FRWY, Suite 1036, DALLAS, TX, 75234, US

(73) Assignee: BIOMAY AG, Vienna (AT)

(21) Appl. No.: 11/815,846

(22) PCT Filed: Feb. 9, 2006
(86) PCT No.: PCT/AT2006/000050

§ 371 Date: Aug. 8, 2007

(30) Foreign Application Priority Data
Feb. 9, 2005 (AT) A 214/2005

(51) Int. Cl. C12Q 1/68; C12Q 1/02; G01N 33/53

(52) U.S. Cl. 435/6; 435/29; 435/7.92; 435/7.1

(57) ABSTRACT

The present invention discloses a method for evaluating the allergen sensitivity of an individual and/or the clinical efficacy of an allergen immunotherapy comprising the steps: providing at least two samples selected from the group consisting of blood or fractions thereof, connective tissue, nasal, bronchial, skin or gut biopsy material from an individual subjected or intended to be subjected to an immunotherapy with at least one pure allergen or derivative thereof, wherein the samples contain cells capable of releasing mediators in response to said allergen, contacting said sample with said allergen or derivative thereof, and determining the amounts of mediators released from said sample and evaluating the allergen sensitivity of the individual prior to therapy and/or the clinical efficacy of the immunotherapy by comparing said amounts.

The present invention relates to methods for monitoring the efficacy of an allergen immunotherapy.

An allergy is an immune malfunction wherein an individual is hypersensitised to react immunologically to typically per se harmless substances called allergens. The principal antibody which is involved in allergic reactions is IgE. Every individual has different IgE antibodies and each allergic substance stimulates production of its own specific IgE. An IgE antibody binding a defined allergen will therefore react only against said allergen. The constant region (Fc region) of IgE is able to bind to specific receptors of cells, which are able to release histamine or other inflammatory mediators, cytokines and/or proteases into the surrounding tissue. Histamine releasing cells are mainly mast and basophilic cells. The release of histamine is initiated when cell-bound IgE is contacted and cross-linked by the allergen.

Especially histamine causes the main allergic reactions. Histamine released in the nose, eyes, and sinuses, for example, stimulates sneezing, a runny nose, and itchy eyes; released in the lungs it causes narrowing and swelling of the lining of the airways and the secretion of thick mucus; in the skin, rashes and hives; and in the digestive system, stomach cramps and diarrhea.

Typical allergens are derived from plant pollens, like rye grass, ragweed, timothy grass and birch trees pollens, mold spores, drugs, like penicillins, sulfonamides, salicylates and local anesthetics, foods, like nuts, seafood, egg, peas, beans, peanuts and other legumes, milk, insect products, like bee-sting venom, wasp sting venom, cockroach calyx and dust mites, and animal hair and dander.

There exists a number of medical treatments for allergies. Mainly three methods are regularly used in medical practice: chemotherapy, immunotherapy and alternative medical methods.

In chemotherapy antagonistic drugs are used to block the action of allergic mediators, preventing activation of cells and degranulation processes. They include antihistamines, cortisone, adrenalin (epinephrine), theophylline and Cromolyn sodium. These drugs help alleviate the symptoms of allergy but play little role in chronic alleviation of the disorder. They can play an imperative role in the acute recovery of someone suffering from anaphylaxis.

In alternative medicine, a number of treatments are considered effective by practitioners in the treatment of allergies, particularly traditional Chinese medicine. However, none of these have been backed up by good quality evidence.

The most promising therapy form is probably immunotherapy. In the course of an immunotherapy where an individual is gradually vaccinated against progressively larger doses of the allergen in question. This can either reduce the severity or eliminate hypersensitivity altogether. Alternatively, monoclonal anti-IgE antibodies may be injected. These antibodies bind to free IgE signalling such sources for destruction. They do not bind to IgE already bound to the Fc receptor on basophils and mast cells as this would stimulate the allergic inflammatory response.

The proteins and glycoproteins used in allergen immunotherapy are usually extracted from materials such as pollens, molds, pelt and insect venoms. Based on the clinical evaluation, repeated subcutaneous injections of a solution of the disease-causing allergen or a derivative thereof are done once or twice a week in increasing doses until a maintenance dose is reached. This maintenance dose is then injected every 2 to 4 weeks.

In order to accomplish an immunotherapy in a successful manner monitoring of the progress of said therapy has to be performed.

For instance, in Wantke et al. (Clin Exp Allergy 23 (1993) 992-995) a method for monitoring an immunotherapy for allergic rhinoconjunctivitis is disclosed. Therein the authors analysed the spontaneous histamine release, i.e., the release without addition of allergen, in patients prior and after the immunotherapy and showed that the histamine release into the blood after exposure to the allergen was significantly reduced after four months of treatment. However, this method cannot be used to assess changes in sensitivity towards a particular allergen and specific efficacy of the treatment.

Stephan et al. (Allergy 44 (1989) 453-459) investigated the effect of bee venom immunotherapy over a period of more than five years by analysing the allergen induced histamine release in whole blood. However, the authors of this study did not correlate the results of histamine release with a clinical parameter, e.g., skin sensitivity and hence no data were shown which would justify to use the assay to measure and reflect clinical sensitivity to a given allergen. Furthermore, no samples obtained before and after treatment were compared among each other.

Yuta et al. (Arerugi 51 (2002) 634-648) studied the histamine release from basophilic cells to evaluate an immunotherapy of allergic rhinitis. The authors analysed samples at the beginning of the treatment and at six months after starting immunotherapy and could show the positive effect of the therapy. In this article samples obtained before and after treatment were analysed and the authors could only show that the rush protocol leads to an exhaustion of the cells but does not show a reduction of histamine release. In this context it should be noted that rush immunotherapy works already before “blocking antibodies” are induced by immunotherapy, i.e., sometimes after hours and few days. This may be interpreted as an exhaustion of cells. However, the assessment of the effect of blocking antibodies which appear after several weeks of treatment is important. Hence an assay where the IgG antibodies are still present, e.g. whole blood, has to be used. In contrast thereto, in Yuta et al. the cells were washed and hence the interference of blocking IgG could not be measured.

In addition to histamine release also other methods for the assessment of basophil and mast cell activation are known, which include measuring the release of leutrienes (Van Rooyen & Anderson, R. J. Immunol. Methods 2004, 288, 1-7), tryptase (Taira M et al., J. Asthma 2002, 39, 315-322) and other mast cell or basophil products which are released upon allergen-specific activation of the mast cells and basophils. Furthermore also the upregulation of activation markers such as CD63 and CD203c resulting from the exposure of an individual to an allergen can be measured by flow cytometry (Hauswirth A. W., et al. J. Allergy Clin. Immunol. 2002, 110, 102-109).

Therefore it is an object of the present invention to provide in vitro means and methods to monitor as close as possible clinical efficacy and the progress of an allergen immunotherapy and allergen sensitivity of an individual.

Therefore the present invention provides a method for evaluating the allergen sensitivity of an individual and/or the clinical efficacy of an allergen immunotherapy comprising the steps:
providing at least two samples selected from the group consisting of blood or fractions thereof, connective tissue, nasal, bronchial, skin or gut biopsy material from an individual subjected or intended to be subjected to an immunotherapy with at least one pure allergen or derivative thereof, wherein the samples contain cells capable of releasing mediators in response to said allergen
contacting said sample with said allergen or derivative thereof, and
determining the amounts of mediators released from said sample and evaluating the allergen sensitivity of the individual prior to therapy and/or the clinical efficacy of the immunotherapy by comparing said amounts.

The evaluation of the allergen sensitivity of an individual and/or the clinical efficacy as well as the progress of an allergen immunotherapy is important in order to guarantee an effective treatment, e.g. by changing the dose and/or time intervals of the administered allergen. Therefore a reliable method to monitor the immunotherapy is required which directly reflects the sensitivity of an individual for a certain type of allergen prion and in the course of on immunotherapy. The measurement of the amount of IgE binding specifically to an allergen turned out to be not suited to determine the degree of sensitisation of an individual for a certain type of allergen, since there is no direct correlation between the amount of IgE present in an individual and the mediator release from mast and basophilic cells. Therefore the release of mediator of a sample of an individual comprising mediator releasing cells is preferred. It was surprisingly found that the method according to the present invention gave comparable, if not identical, results as the traditionally used skin sensitivity test.

The samples provided by an individual are preferably contacted with the same allergen, which is used for immunotherapy. However, it is also possible to perform the immunotherapy with an allergen extract and to monitor said therapy with substantially purified (“pure”) allergens.

Of course the method according to the present invention may also be used to monitor the progress of an allergen immunotherapy by determining the allergen sensitivity of an individual in the course of the therapy.

“Allergens” according to the present invention are molecules or mixtures of molecules able to induce the production of specific antibodies (IgE) which are responsible to trigger mediator release of a mediator releasing cell and to cause consequently allergic effects in the individual. Of course, “allergens” are also capable to induce the production of antibodies other than IgE (e.g. IgG). However, the allergens used in the method according to the present invention are preferably purified, i.e. the allergens consist substantially of one single allergen molecule, whereby the degree of purity exceeds 90% (w/w), preferably 95% (w/w), most preferably 99% (w/w). Due to the use of substantially purified or isolated allergens it is possible to determine and to dose in a reproducible manner the amount of allergen used in immunotherapy as well as used in a method according to the present invention. In contrast thereto allergen extracts contain varying concentrations of the specific allergen, depending on the specific purification conditions. Furthermore allergen extracts may also contain more than one allergen, which may be present in the extract in different concentrations (the amount of the allergen of interest is not definable in an accurate manner) and may further provoke cross reactions (see for instance Marth K et al. (2004) J. Allergy Clin. Immunol. 113: 470-474; Marth K et al. (2004) XXIII EAACI congress abstract book 597: 181; Akkerdaas H J et al. (2003) Arb. Paul Ehrlich Inst. Bundesamt Sera Impfstoffe Frankf. a. M. 94: 87-95). In addition, allergen extracts may contain contaminations or substance which may influence the stability of the extract. This problem can also be avoided by using substantially purified or “pure” allergens.

The term “derivative” allergen as used herein refers to modified (deleted, point mutated, truncated etc.) allergens which still exhibit the same antigenic and IgE binding properties as the native allergen from which they are derived from.

According to a preferred embodiment of the present invention the mediators are selected from the group consisting of histamine, tryptase, prostaglandins, leukotrienes, especially cysteinyl leukotrienes, eosinophil cationic protein, cytokines, like interleukins (IL), especially IL-2R, CD63, CD203c and combinations thereof.

The allergic response of an individual after the exposure of said individual to an allergen is primarily caused by the release of mediators by mast cells. These mediators produce the early symptoms of an allergic reaction (e.g. sneezing, itching) and stimulate the production and infiltration into local tissue of circulating leukocytes (e.g. eosinophils). The mediators can be released from the cells by degranulation (histamine and proteases) or after neosynthesis of said mediators (Quraishi S. A. et al., JAOA Supplement 5, 104:S7-S15). According to the present invention also activation markers—besides mediators—can be determined (e.g. Yoshimura C., et al., (2002) J Allergy Clin Immunol. 109:817-23).

The sample is blood or fractions thereof (e.g. plasma, serum), connective tissue, nasal, bronchial, skin or gut biopsy material.

Mediator releasing cells can be found in blood and fractions thereof, in connective and several other tissues. It was surprisingly found that the method according to the present invention closely mirrors cutaneous sensitivity when using pure allergens, especially when whole blood is used. In contrast thereto, measurements of specific IgE did not correlate with cutaneous sensitivity. Therefore the sample to be used in a method according to the present invention may be a blood sample (preferably heparinised blood) or connective tissue.

The mediator releasing cells used in the method according to the present invention may be isolated from the sample. Due to this isolation other possibly disturbing substances present in the sample may be removed. Especially considering that blood, for instance, may contain released mediator providing a high background during the determination of the amount of mediator released into the sample upon contact with an allergen. This problem may be avoided by measuring the amount of mediator present in the sample prior its exposure to the allergen. On the other hand experimental data revealed that substantially no correlation between histamine release and skin sensitivity, for instance, exists. Therefore, the samples to be used according to the present invention are not isolated or washed prior contacting the sample with the allergen or derivative thereof. This may be reasoned by the fact that when mediator releasing cells are washed all antibodies including those IgG antibodies which should be induced in the course of an allergen therapy and which would act as blocking antibodies in order to reduce the amount of IgE-allergen complexes (due to competition with IgE molecules) in the sample are removed (see e.g. Stahl-Skov et al. (1977) Clin. Exp. Immunol. 27: 432-439)

Preferably said cells are mast and/or basophilic and/or eosinophilic cells.

Mast and basophilic cells are those cells which release most of the mediators, especially histamine, when exposed to an allergen. Mast cells are found in connective tissues of the skin, lung and gastrointestinal tract, whereas basophilic cells are found in blood. These cells can be isolated by known methods and be used in a method according to the present invention. Isolation protocols for mast cells can be found in Jamur M C et al. (J Histochem Cytochem. 1997 45:1715-1722), Massey W A (J. Immunol. 1991 147:1621-7), isolation protocols for basophilic cells in Valent P. (Proc. Natl. Acad. Sci USA 1989, 86, 5542-5546).

According to a preferred embodiment of the present invention the sample further comprises immunoglobulins (Ig), especially immunoglobulin G (IgG).

The procedure should preferably be carried out with samples containing IgG, e.g. whole blood samples. The presence of IgG in such samples is preferred since it allows the measurement of the interference of blocking IgG during the exposure of said cells to the allergen. In the course of an allergen immunotherapy IgGs directed to said allergen are produced. These IgGs bind to the allergen when an individual is contacted with said allergen and prevent that the allergen binds to IgE. Since the production of allergen binding IgGs is therefore directly involved in the response of an individual to an allergen and thus influencing the allergen sensitivity of an individual, the sample should preferably contain IgGs.

In order to evaluate the allergen sensitivity of an individual or the clinical efficacy of an allergen immunotherapy the samples are preferably provided before and after subjecting said individual to an immunotherapy.

To monitor and to evaluate the efficacy of an immunotherapy it is necessary to determine the sensitivity of an individual to an allergen prior and in the course of the therapy. Therefore the mediator release is determined at various stages of the therapy. In the course of the therapy the sensitivity to an allergen ideally decreases. Furthermore, the determination of the mediator release at one or more time points before the immunotherapy may be useful for dosing the allergen in the course of the therapy.

According to another preferred embodiment of the present invention the samples are provided after subjecting said individual to an immunotherapy.

Of course an immunotherapy may also be evaluated solely by analysing samples after the first administration of a medicament comprising an allergen.

Preferably the at least one sample is provided after a maximum of 1 hour, 2 hours, 6 hours, 12 hours, 24 hours, 5 days, 10 days, 4 weeks, 6 months, 12 months, 24 months and 36 months, after subjecting said individual to an immunotherapy.

The sample to be analysed may be provided after a defined time period after the first administration of the allergen. Also the time intervals in between the single determinations of the mediator release may be preferably varied within the range of 1 hour, 2 hours, 6 hours, 12 hours, 2 days, 5 days, 1 week, 2 weeks, 4 weeks, 2 months, 4 months, 6 months, 12 months and 24 months.

According to a preferred embodiment said allergen is recombinantly produced.

An efficient allergen immunotherapy and an accurate method to determine the release of mediator is preferably conducted with an allergen, which is recombinantly produced. Due to genetic engineering it is possible to produce a specific allergen in a high amount and to isolate said allergen. Allergens are usually isolated directly from the source which contains the allergen (e.g. pollen) and since the allergen is contained in an extract, said allergen is always part of a mixture of different allergenic and potential allergenic substances. Even purified “natural allergens” consist of several isoforms, some of them which may be even hypo or non-allergenic and hence give false test results (Ferreira F., et al., J. Exp. Med. 1996, 183, 599-609). This problem can be avoided by the recombinant production of allergens. The allergen used for the administration to an individual may also be used in a method according to the present invention.

Said allergen comprises preferably at least one deletion, at least one substitution or at least one insertion.

Also hypoallergenic allergen or derivatives thereof can be used when it comes to the question whether the patient may become sensitised to these derivatives during treatment.

According to a preferred embodiment of the present invention said allergen is modified by reshuffling the fragments of said allergen by genetic engineering.

The sample is preferably contacted with varying concentrations of said allergen.

The amount of mediator released from a mediator releasing cell depends on the concentration of the allergen employed in the method according to the present invention. The higher the concentration of the allergen used to induce the release a distinct amount of mediator is, the lower is the sensitivity of the cells provided from an individual and vice versa. Therefore the determination of the amount of mediator released requires the use of varying concentrations of allergen.

Preferably the concentration of said allergen is selected within the range of 1 ng/ml to 100 μg/ml, preferably within the range of 1 pg/ml to 10 μg/ml.

According to a preferred embodiment the total amount of mediator of the cells contained in the sample provided by an individual is determined.

In order to determine the amount of total mediator present in the cells, these cells are lysed e.g. by several thawing and freezing cycles. The determined amount of mediator indicates the mediator potentially releasable by said cells, which value may be employed to determining the degree of cellular sensitisation of the cells to a certain allergen.

A degree of cellular sensitisation is preferably defined by determining the concentration of said allergen inducing the release of 10%, preferably 30%, of the total amount of mediator of said cells.

The degree of cellular sensitisation is an indicator of the progress of the immunotherapy because it reveals the concentration, at which a cell releases 10%, preferably 20%, 25%, 30%, of the total amount of mediator present in the mediator releasing cell. In the course of a successful allergen immunotherapy the concentration of the allergen employed should increase because a high concentration of allergen releasing a certain amount of mediator from said cells indicates that the cells are less sensitive than in a previous measurement. Also the dose inducing maximum release of the mediator may be evaluated. This allows to create a dose response curve and to measure the shifting of said curve in the course of an allergen immunotherapy.

Therefore, the allergen sensitivity of an individual and/or the clinical efficacy of the allergen immunotherapy is preferably evaluated by observing the degree of cellular sensitisation in the course of said immunotherapy.

According to a preferred embodiment of the present invention the mediator in the sample is determined by an immunological and/or a chromatographical method, preferably the method is selected from the group consisting of radioimmunoassay (RIA), enzyme linked immunosorbent assay (ELISA), high performance liquid chromatography (HPLC), reverse transcriptase polymerase chain reaction, immunofluorescence flow cytometry and combinations thereof.

All of these methods have been established to come closer to clinical sensitivity. However, none of these methods has been used to look at a pure allergen in serology, basophil activation and skin sensitivity (e.g. Pierkes M. et al., J Allergy Clin Immunol. (1999) 103:326-32; Di Lorenzo G. et al., J Allergy Clin Immunol. (1997) 100:832-7).

Preferred allergens to be used by the present invention include all major protein allergens available e.g. under www.allergen.org/List.htm. Specifically preferred groups of allergens according to the present invention include major allergens such as major birch pollen allergens, e.g. Bet v 1, major timothy grass pollen allergens, e.g. Phl p 1, Phl p 2, Phl p 5 and Phl p 6, major house dust mite allergens, e.g. Der p 1, Der p 2, major cat allergen, e.g. Fel d 1, major bee and wasp allergens (see list), other profilins, especially Phl p 12, other birch allergens, especially Bet v 4, storage mite allergens, especially Lep d 2, and the allergens listed in table 1.

TABLE 1
preferred allergen to be used by the present invention
(including reference examples)
ALLERGENS

| | | Biochem.ID or | | cDNA or | Reference, |
|---|---|---|---|---|---|
| Species Name | Allergen Name | Obsolete name | Mw | protein | Acc. No. |
| Ambrosia artemisiifolia | Amb a 1 | antigen E | 8 | C | 8, 20 |
| short ragweed | Amb a 2 | antigen K | 38 | C | 8, 21 |
| | Amb a 3 | Ra3 | 11 | C | 22 |
| | Amb a 5 | Ra5 | 5 | C | 11, 23 |
| | Amb a 6 | Ra6 | 10 | C | 24, 25 |
| | Amb a 7 | Ra7 | 12 | P | 26 |
| Ambrosia trifida | Amb t 5 | Ra5G | 4.4 | C | 9, 10, 27 |
| giant ragweed | | | | | |
| Artemisia vulgaris | Art v 1 | | 27-29 | C | 28 |
| mugwort | Art v 2 | | 35 | P | 28A |
| | Art v 3 | lipid transfer protein | 12 | P | 53 |
| | Art v 4 | profilin | 14 | C | 29 |
| Helianthus annuus | Hel a 1 | | 34 | | 29A |
| sunflower | Hel a 2 | profilin | 15.7 | C | Y15210 |
| Mercurialis annua | Mer a 1 | profilin | 14-15 | C | Y13271 |
| Caryophyllales | | | | | |
| Chenopodium album | Che a 1 | | 17 | C | AY049012, 29B |
| lamb's-quarters, pigweed, | Che a 2 | profilin | 14 | C | AY082337 |
| white goosefoot | Che a 3 | polcalcin | 10 | C | AY082338 |
| Salsola kali | Sal k 1 | | 43 | P | 29C |
| Russian-thistle | | | | | |
| Rosales | | | | | |
| Humulus japonicus | Hum j 4w | | | C | AY335187 |
| Japanese hop | | | | | |
| Parietaria judaica | Par j 1 | lipid transfer protein 1 | 15 | C | see list of isoallergens |
| | Par j 2 | lipid transfer protein 2 | | C | see list of isoallergens |
| | Par j 3 | profilin | | C | see list of isoallergens |
| Parietaria officinalis | Par o 1 | lipid transfer protein | 15 | | 29D |

B. Grasses

| | | | | | |
|---|---|---|---|---|---|
| Poales | | | | | |
| Cynodon dactylon | Cyn d 1 | | 32 | C | 30, S83343 |
| Bermuda grass | Cyn d 7 | | | C | 31, X91256 |
| | Cyn d 12 | profilin | 14 | C | 31a, Y08390 |
| | Cyn d 15 | | 9 | C | AF517686 |
| | Cyn d 22w | enolase | data | | pending |
| | Cyn d 23 | Cyn d 14 | 9 | C | AF517685 |
| | Cyn d 24 | Pathogenesis-related p. | 21 | P | pending |
| Dactylis glomerata | Dac g 1 | AgDg1 | 32 | P | 32 |
| orchard grass | Dac g 2 | | 11 | C | 33, S45354 |
| | Dac g 3 | | | C | 33A, U25343 |
| | Dac g 5 | | 31 | P | 34 |
| Festuca pratensis | Fes p 4w | | 60 | — | |
| meadow fescue | | | | | |
| Holcus lanatus | Hol l 1 | | | C | Z27084 |
| velvet grass | | | | | |
| Lolium perenne | Lol p 1 | group I | 27 | C | 35, 36 |
| rye grass | Lol p 2 | group II | 11 | P | 37, 37A, X73363 |
| | Lol p 3 | group III | 11 | P | 38 |
| | Lol p 5 | Lol p IX, Lol p Ib | 31/35 | C | 34, 39 |
| | Lol p 11 | hom: trypsin inhibitor | 16 | | 39A |
| Phalaris aquatica | Pha a 1 | | | C | 40, S80654 |
| canary grass | | | | | |
| Phleum pratense | Phl p 1 | | 27 | C | X78813 |
| timothy | Phl p 2 | | | C | X75925, 41 |
| | Phl p 4 | | | P | 41A |
| | Phl p 5 | Ag25 | 32 | C | 42 |
| | Phl p 6 | | | C | Z27082, 43 |
| | Phl p 11 | trypsin inhibitor hom. | 20 | C | AF521563, 43A |
| | Phl p 12 | profilin | | C | X77583, 44 |
| | Phl p 13 | polygalacturonase | 55-60 | C | AJ238848 |
| Poa pratensis | Poa p 1 | group I | 33 | P | 46 |
| Kentucky blue grass | Poa p 5 | | 31/34 | C | 34, 47 |
| Sorghum halepense | Sor h 1 | | | C | 48 |
| Johnson grass | | | | | |

C. Trees

| | | | | | |
|---|---|---|---|---|---|
| Arecales | | | | | |
| Phoenix dactylifera | Pho d 2 | profilin | 14.3 | C | Asturias p.c. |
| date palm | | | | | |
| Fagales | | | | | |
| Alnus glutinosa | Aln g 1 | | 17 | C | S50892 |
| alder | | | | | |
| Betula verrucosa | Bet v 1 | | 17 | C | see list of isoallergens |
| birch | Bet v 2 | profilin | 15 | C | M65179 |
| | Bet v 3 | | | C | X79267 |
| | Bet v 4 | | 8 | C | X87153, S54819 |
| | Bet v 6 | h: isoflavone reductase | 33.5 | C | see list of isoallergens |
| | Bet v 7 | cyclophilin | 18 | P | P81531 |
| Carpinus betulus | Car b 1 | | 17 | C | see list of isoallergens |
| hornbeam | | | | | |
| Castanea sativa | Cas s 1 | | 22 | P | 52 |
| chestnut | Cas s 5 | chitinase | | | |
| | Cas s 8 | lipid transfer protein | 9.7 | P | 53 |
| Corylus avellana | Cor a 1 | | 17 | C | see list of isoallergens |
| hazel | Cor a 2 | profilin | 14 | C | |
| | Cor a 8 | lipid transfer protein | 9 | C | |
| | Cor a 9 | 11S globulin-like protein | 40/? | C | Beyer p.c. |
| | Cor a 10 | luminal binding prot. | 70 | C | AY295617 |
| | Cor a 11 | 7S vicilin-like prot. | 48 | C | AF441864 |
| Quercus alba | Que a 1 | | 17 | P | 54 |
| White oak | | | | | |
| Lamiales | | | | | |
| Oleaceae | | | | | |
| Fraxinus excelsior | Fra e 1 | | 20 | P | 58A, AF526295 |
| ash | | | | | |
| Ligustrum vulgare | Lig v 1 | | 20 | P | 58A |
| privet | | | | | |
| Olea europea | Ole e 1 | | 16 | C | 59, 60 |
| olive | Ole e 2 | profilin | 15-18 | C | 60A |
| | Ole e 3 | | 9.2 | | 60B |
| | Ole e 4 | | 32 | P | P80741 |
| | Ole e 5 | superoxide dismutase | 16 | P | P80740 |
| | Ole e 6 | | 10 | C | 60C, U86342 |
| | Ole e 7 | | ? | P | 60D, P81430 |
| | Ole e 8 | Ca2+-binding protein | 21 | C | 60E, AF078679 |
| | Ole e 9 | beta-1,3-glucanase | 46 | C | AF249675 |
| | Ole e 10 | glycosyl hydrolase hom. | 11 | C | 60F, AY082335 |
| Syringa vulgaris | Syr v 1 | | 20 | P | 58A |
| lilac | | | | | |
| Plantaginaceae | Pla 1 1 | | 18 | P | P842242 |
| Plantago lanceolata | | | | | |
| English plantain | | | | | |
| Pinales | | | | | |
| Cryptomeria japonica | Cry j 1 | | 41-45 | C | 55, 56 |
| sugi | Cry j 2 | | | C | 57, D29772 |
| Cupressus arisonica | Cup a 1 | | 43 | C | A1243570 |
| cypress | | | | | |
| Cupressus sempervirens | Cup s 1 | | 43 | C | see list of isoallergens |
| common cypress | Cup s 3w | | 34 | C | ref pending |
| Juniperus ashei | Jun a 1 | | 43 | P | P81294 |
| mountain cedar | Jun a 2 | | | C | 57A, AJ404653 |
| | Jun a 3 | | 30 | P | 57B, P81295 |
| Juniperus oxycedrus | Jun o 4 | hom: calmodulin | 29 | C | 57C, AF031471 |
| prickly juniper | | | | | |
| Juniperus sabinoides | Jun s 1 | | 50 | P | 58 |
| mountain ceder | | | | | |
| Juniperus virginiana | Jun v 1 | | 43 | P | P81825, 58B |
| eastern red cedar | | | | | |
| Platanaceae | Pla a 1 | | 18 | P | P82817 |
| Platanus acerifolia | Pla a 2 | | 43 | P | P82967 |
| London plane tree | Pla a 3 | lipid transfer protein | 10 | P | Iris p.c. |

D. Mites

| | | | | | |
|---|---|---|---|---|---|
| Acarus siro | Aca s 13 | arthropod | 14* | C | AJ006774 |
| mite | | fatty acid binding prot. | | | |
| Blomia tropicalis | Blo t 1 | cysteine protease | 39 | C | AF277840 |
| mite | Blo t 3 | trypsin | 24* | C | Cheong p.c. |
| | Blo t 4 | alpha amylase | 56 | C | Cheong p.c. |
| | Blo t 5 | | | C | U59102 |
| | Blo t 6 | chymotrypsin | 25 | C | Cheong p.c. |
| | Blo t 10 | tropomyosin | 33 | C | 61 |
| | Blo t 11 | paramyosin | 110 | C | AF525465, 61A |
| | Blo t 12 | Bt11a | | C | U27479 |
| | Blo t 13 | Bt6, fatty acid bind prot. | | C | U58106 |
| | Blo t 19 | anti-microbial pep. hom. | 7.2 | C | Cheong p.c. |
| Dermatophagoides farinae | Der f 1 | cysteine protease | 25 | C | 69 |
| American house dust mite | Der f 2 | | 14 | C | 70, 70A, see list of |
| | | | | | isoallergens |
| | Der f 3 | trypsin | 30 | C | 63 |
| | Der f 7 | | 24-31 | C | SW: Q26456, 71 |
| | Der f 10 | tropomyosin | | C | 72 |
| | Der f 11 | paramyosin | 98 | C | 72A |
| | Der f 14 | mag3, apolipophorin | | C | D17686 |
| | Der f 15 | 98k chitinase | 98 | C | AF178772 |
| | Der f 16 | gelsolin/villin | 53 | C | 71A |
| | Der f 17 | Ca binding EF protein | 53 | C | 71A |
| | Der f 18w | 60k chitinase | 60 | C | Weber p.c. |
| Dermatophagoides microceras | Der m 1 | cysteine protease | 25 | P | 68 |
| house dust mite | | | | | |
| Dermatophagoides pteronyssinus | Der p 1 | antigen P1, cysteine protease | 25 | C | 62, see list of |
| European house dust mite | | | | | isoallergens |
| | Der p 2 | | 14 | C | 62A-C, see list of |
| | | | | | isoallergens |
| | Der p 3 | trypsin | 28/30 | C | 63 |
| | Der p 4 | amylase | 60 | P | 64 |
| | Der p 5 | | 14 | C | 65 |
| | Der p 6 | chymotrypsin | 25 | P | 66 |
| | Der p 7 | | 22/28 | C | 67 |
| | Der p 8 | glutathione transferase | | C | 67A |
| | Der p 9 | collagenolytic serine pro. | | P | 67B |
| | Der p 10 | tropomyosin | 36 | C | Y14906 |
| | Der p 14 | apolipophorin like prot. | | C | Epton p.c. |
| Euroglyphus maynei | Eur m 2 | | | C | see list of isoallergens |
| mite | Eur m 14 | apolipophorin | 177 | C | AF149827 |
| Glycyphagus domesticus | Gly d 2 | | | C | 72B, see isoallergen |
| storage mite | | | | | list |
| Lepidoglyphus destructor | Lep d 2 Lep d 1 | | 15 | C | 73, 74, 74A, see |
| storage mite | | | | | isoallergen list |
| | Lep d 5 | | | C | 75, AJ250278 |
| | Lep d 7 | | | C | 75, AJ271058 |
| | Lep d 10 | tropomyosin | | C | 75A, AJ250096 |
| | Lep d 13 | | | C | 75, AJ250279 |
| Tyrophagus putrescentiae | Tyr p 2 | | | C | 75B, Y12690 |
| storage mite | | | | | |

E. Animals

| | | | | | |
|---|---|---|---|---|---|
| Bos domesticus | Bos d 2 | Ag3, lipocalin | 20 | C | 76, see isoallergen |
| domestic cattle | | | | | list |
| (see also foods) | Bos d 3 | Ca-binding S100 hom. | 11 | C | L39834 |
| | Bos d 4 | alpha-lactalbumin | 14.2 | C | M18780 |
| | Bos d 5 | beta-lactoglobulin | 18.3 | C | X14712 |
| | Bos d 6 | serum albumin | 67 | C | M73993 |
| | Bos d 7 | immunoglobulin | 160 | | 77 |
| | Bos d 8 | caseins | 20-30 | | 77 |
| Canis familiaris | Can f 1 | | 25 | C | 78, 79 |
| (Canis domesticus) | Can f 2 | | 27 | C | 78, 79 |
| dog | Can f 3 | albumin | | C | S72946 |
| | Can f 4 | | 18 | P | A59491 |
| Equus caballus | Equ c 1 | lipocalin | 25 | C | U70823 |
| domestic horse | Equ c 2 | lipocalin | 18.5 | P | 79A, 79B |
| | Equ c 3 | Ag3-albumin | 67 | C | 79C, X74045 |
| | Equ c 4 | | 17 | P | 79D |
| | Equ c 5 | AgX | 17 | P | Goubran Botros p.c. |
| Felis domesticus | Fel d 1 | cat-1 | 38 | C | 15 |
| cat (saliva) | Fel d 2 | albumin | | C | 79E, X84842 |
| | Fel d 3 | cystatin | 11 | C | 79F, AF238998 |
| | Fel d 4 | lipocalin | 22 | C | AY497902 |
| | Fel d 5w | immunoglobulin A | 400 | | Adedoyin p.c. |
| | Fel d 6w | immunoglobulin M | 800-1000 | | Adedoyin p.c. |
| | Fel d 7w | immunoglobulin G | 150 | | Adedoyin p.c. |
| Cavia porcellus | Cav p 1 | lipocalin homologue | 20 | P | SW: P83507, 80 |
| guinea pig | Cav p 2 | | 17 | P | SW: P83508 |
| Mus musculus | Mus m 1 | MUP | 19 | C | 81, 81A |
| mouse (urine) | | | | | |
| Rattus norvegius | Rat n 1 | | 17 | C | 82, 83 |
| rat (urine) | | | | | |

F. Fungi (moulds)

| | | | | | |
|---|---|---|---|---|---|
| 1. Ascomycota | | | | | |
| 1.1 Dothideales | | | | | |
| Alternaria alternata | Alt a 1 | | 28 | C | U82633 |
| | Alt a 2 | | 25 | C | 83A, U62442 |
| | Alt a 3 | heat shock prot. | 70 | C | U87807, U87808 |
| | Alt a 4 | prot. disulfideisomerase | 57 | C | X84217 |
| | Alt a 6 | acid ribosomal prot. P2 | 11 | C | X78222, U87806 |
| | Alt a 7 | YCP4 protein | 22 | C | X78225 |
| | Alt a 10 | aldehyde dehydrogenase | 53 | C | X78227, P42041 |
| | Alt a 11 | enolase | 45 | C | U82437 |
| | Alt a 12 | acid ribosomal prot. P1 | 11 | C | X84216 |
| Cladosporium herbarum | Cla h 1 | | 13 | | 83B, 83C |
| | Cla h 2 | | 23 | | 83B, 83C |
| | Cla h 3 | aldehyde dehydrogenase | 53 | C | X78228 |
| | Cla h 4 | acid ribosomal prot. P2 | 11 | C | X78223 |
| | Cla h 5 | YCP4 protein | 22 | C | X78224 |
| | Cla h 6 | enolase | 46 | C | X78226 |
| | Cla h 12 | acid ribosomal prot. P1 | 11 | C | X85180 |
| 1.2 Eurotiales | | | | | |
| Aspergillus flavus | Asp fl 13 | alkaline serine protease | 34 | | 84 |
| Aspergillus fumigatus | Asp f 1 | | 18 | C | M83781, S39330 |
| | Asp f 2 | | 37 | C | U56938 |
| | Asp f 3 | peroxisomal protein | 19 | C | U20722 |
| | Asp f 4 | | 30 | C | AJ001732 |
| | Asp f 5 | metalloprotease | 40 | C | Z30424 |
| | Asp f 6 | Mn superoxide dismut. | 26.5 | C | U53561 |
| | Asp f 7 | | 12 | C | AJ223315 |
| | Asp f 8 | ribosomal prot. P2 | 11 | C | AJ224333 |
| | Asp f 9 | | 34 | C | AJ223327 |
| | Asp f 10 | aspartic protease | 34 | C | X85092 |
| | Asp f 11 | peptidyl-prolyl isomeras | 24 | | 84A |
| | Asp f 12 | heat shock prot. P90 | 90 | C | 85 |
| | Asp f 13 | alkaline serine protease | 34 | | 84B |
| | Asp f 15 | | 16 | C | AJ002026 |
| | Asp f 16 | | 43 | C | g3643813 |
| | Asp f 17 | | | C | AJ224865 |
| | Asp f 18 | vacuolar sarine protease | 34 | | 84C |
| | Asp f 22w | enolase | 46 | C | AF284645 |
| | Asp f 23 | L3 ribosomal protein | 44 | C | 85A, AF464911 |
| Aspergillus niger | Asp n 14 | bata-xylosidase | 105 | C | AF108944 |
| | Asp n 18 | vacuolar serine protease | 34 | C | 84B |
| | Asp n 25 | 3-phytase B | 66-100 | C | 85B, P34754 |
| | Asp n ? | | 85 | C | Z84377 |
| Aspergillus oryzae | Asp o 13 | alkaline serine protease | 34 | C | X17561 |
| | Asp o 21 | TAKA-amylase A | 53 | C | D00434, M33218 |
| Penicillium brevicompactum | Pen b 13 | alkaline serine protease | 33 | | 86A |
| Penicillium chrysogenum | Pen ch 13 | alkaline serine protease | 34 | | 87 |
| (formerly P. notatum) | Pen ch 18 | vacuolar serine protease | 32 | | 87 |
| | Pen ch 20 | N-acetyl glucosaminidas | 68 | | 87A |
| Penicillium citrinum | Pen c 3 | peroxisomal mem. prot. | 18 | | 86B |
| | Pen c 13 | alkaline serine protease | 33 | | 86A |
| | Pen c 19 | heat shock prot. P70 | 70 | C | U64207 |
| | Pen c 22w | enolase | 46 | C | AF254643 |
| | Pen c 24 | elongation factor 1 beta | | C | AY363911 |
| Penicillium oxalicum | Pen o 18 | vacuolar serine protease | 34 | | 87B |
| 1.3 Hypocreales | | | | | |
| Fusarium culmorum | Fus c 1 | ribosomal prot. P2 | 11* | C | AY077706 |
| | Fus c 2 | thioredoxin-like prot. | 13* | C | AY077707 |
| 1.4 Onygenales | | | | | |
| Trichophyton rubrum | Tri r 2 | | | C | 88 |
| | Tri r 4 | serine protease | | C | 88 |
| Trichophyton tonsurans | Tri t 1 | | 30 | P | 88A |
| | Tri t 4 | serine protease | 83 | C | 88 |
| 1.5 Saccharomycetales | | | | | |
| Candida albicans | Cand a 1 | | 40 | C | 89 |
| | Cand a 3 | peroxisomal protein | 29 | C | AY136739 |
| Candida boidinii | Cand b 2 | | 20 | C | J04984, J04985 |
| 2. Basidiomycotina | | | | | |
| 2.1 Hymenomycetes | | | | | |
| Psilocybe cubensis | Psi c 1 | | | | |
| | Psi c 2 | cyclophilin | 16 | | 89A |
| Coprinus comatus | Cop c 1 | leucine zipper protein | 11 | C | AJ132235 |
| shaggy cap | Cop c 2 | | | | AJ242791 |
| | Cop c 3 | | | | AJ242792 |
| | Cop c 5 | | | | AJ242793 |
| | Cop c 7 | | | | AJ242794 |
| 2.2 Urediniomycetes | | | | | |
| Rhodotorula mucilaginosa | Rho m 1 | enolase | 47 | C | 89B |
| | Rho m 2 | vacuolar serine protease | 31 | C | AY547285 |
| 2.3 Ustilaginomycetes | | | | | |
| Malassezia furfur | Mala f 2 | MF1, peroxisomal | 21 | C | AB011804, 90 |
| | | membrane protein | | | |
| | Mala f 3 | MF2, peroxisomal | 20 | C | AB011805, 90 |
| | | membrane protein | | | |
| | Mala f 4 | mitochondrial malate | 35 | C | AF084828, 90A |
| | | dehydrogenase | | | |
| Malassezia sympodialis | Mala s 1 | | | C | X96486, 91 |
| | Mala s 5 | | 18* | C | AJ011955 |
| | Mala s 6 | | 17* | C | AJ011956 |
| | Mala s 7 | | | C | AJ011957, 91A |
| | Mala s 8 | | 19* | C | AJ011958, 91A |
| | Mala s 9 | | 37* | C | AJ011959, 91A |
| | Mala s 10 | heat shock prot. 70 | 86 | C | AJ428052 |
| | Mala s 11 | Mn superoxide dismut. | 23 | C | AJ548421 |
| 3. Deuteromycotina | | | | | |
| 3.1 Tuberculariales | | | | | |
| Epicoccum purpurascens | Epi p 1 | serine protease | 30 | P | SW: P83340, 91B |
| (formerly E. nigrum) | | | | | |

G. Insects

| | | | | | |
|---|---|---|---|---|---|
| Aedes aegyptii | Aed a 1 | apyrase | 68 | C | L12389 |
| mosquito | Aed a 2 | | 37 | C | M33157 |
| Apis mellifera | Api m 1 | phospholipase A2 | 16 | C | 92 |
| honey bee | Api m 2 | hyaluronidase | 44 | C | 93 |
| | Api m 4 | melittin | 3 | C | 94 |
| | Api m 6 | | 7-8 | P | Kettner p.c. |
| | Api m 7 | CUB serine protease | 39 | C | AY127579 |
| Bombus pennsylvanicus | Bom p 1 | phospholipase | 16 | P | 95 |
| bumble bee | Bom p 4 | protease | | P | 95 |
| Blattella germanica | Bla g 1 | Bd90k | | C | |
| German cockroach | Bla g 2 | aspartic protease | 36 | C | 96 |
| | Bla g 4 | calycin | 21 | C | 97 |
| | Bla g 5 | glutathione transferase | 22 | C | 98 |
| | Bla g 6 | troponin C | 27 | C | 98 |
| Periplaneta americana | Per a 1 | Cr-PII | | C | |
| American cockroach | Per a 3 | Cr-PI | 72-78 | C | 98A |
| | Per a 7 | tropomyosin | 37 | C | Y14854 |
| Chironomus kiiensis | Chi k 10 | tropomyosin | 32.5* | C | AJ012184 |
| midge | | | | | |
| Chironomus thummi thummi | Chi t 1-9 | hemoglobin | 16 | C | 99 |
| midge | Chi t 1.01 | component III | 16 | C | P02229 |
| | Chi t 1.02 | component IV | 16 | C | P02230 |
| | Chi t 2.0101 | component I | 16 | C | P02221 |
| | Chi t 2.0102 | component IA | 16 | C | P02221 |
| | Chi t 3 | component II-beta | 16 | C | P02222 |
| | Chi t 4 | component IIIA | 16 | C | P02231 |
| | Chi t 5 | component VI | 16 | C | P02224 |
| | Chi t 6.01 | component VIIA | 16 | C | P02226 |
| | Chi t 6.02 | component IX | 16 | C | P02223 |
| | Chi t 7 | component VIIB | 16 | C | P02225 |
| | Chi t 8 | component VIII | 16 | C | P02227 |
| | Chi t 9 | component X | 16 | C | P02228 |
| Ctenocephalides felis felis | Cte f 1 | | | | |
| cat flea | Cte f 2 | M1b | 27 | C | AF231352 |
| | Cte f 3 | | 25 | C | |
| Thaumetopoea pityocampa | Tha p 1 | | 15 | P | PIR: A59396, 99A |
| pine processionary moth | | | | | |
| Lepisma saccharina | Lep s 1 | tropomyosin | 36 | C | AJ309202 |
| silverfish | | | | | |
| Dolichovespula maculata | Dol m 1 | phospholipase A1 | 35 | C | 100 |
| white face hornet | Dol m 2 | hyaluronidase | 44 | C | 101 |
| | Dol m 5 | antigen 5 | 23 | C | 102, 103 |
| Dolichovespula arenaria | Dol a 5 | antigen 5 | 23 | C | 104 |
| yellow hornet | | | | | |
| Polistes annularies | Pol a 1 | phospholipase A1 | 35 | P | 105 |
| wasp | Pol a 2 | hyaluronidase | 44 | P | 105 |
| | Pol a 5 | antigen 5 | 23 | C | 104 |
| Polistes dominulus | Pol d 1 | | | | Hoffman p.c. |
| Mediterranean paper wasp | Pol d 4 | serine protease | 32-34 | C | Hoffman p.c. |
| | Pol d 5 | | | | P81656 |
| Polistes exclamans | Pol e 1 | phospholipase A1 | 34 | P | 107 |
| wasp | Pol e 5 | antigen 5 | 23 | C | 104 |
| Polistes fuscatus | Pol f 5 | antigen 5 | 23 | C | 106 |
| wasp | | | | | |
| Polistes gallicus | Pol g 5 | antigen 5 | 24 | C | P83377 |
| wasp | | | | | |
| Polistes metricus | Pol m 5 | antigen 5 | 23 | C | 106 |
| wasp | | | | | |
| Vespa crabo | Vesp c 1 | phospholipase | 34 | P | 107 |
| European hornet | Vesp c 5 | antigen 5 | 23 | C | 106 |
| Vespa mandarina | Vesp m 1 | | | | Hoffman p.c. |
| giant asian hornet | Vesp m 5 | | | | P81657 |
| Vespula flavopilosa | Ves f 5 | antigen 5 | 23 | C | 106 |
| yellowjacket | | | | | |
| Vespula germanica | Ves g 5 | antigen 5 | 23 | C | 106 |
| yellowjacket | | | | | |
| Vespula maculifrons | Ves m 1 | phospholipase A1 | 33.5 | C | 108 |
| yellowjacket | Ves m 2 | hyaluronidase | 44 | P | 109 |
| | Ves m 5 | antigen 5 | 23 | C | 104 |
| Vespula pennsylvanica | Ves p 5 | antigen 5 | 23 | C | 106 |
| yellowjacket | | | | | |
| Vespula squamosa | Ves s 5 | antigen 5 | 23 | C | 106 |
| yellowjacket | | | | | |
| Vespula vidua | Ves vi 5 | antigen 5 | 23 | C | 106 |
| wasp | | | | | |
| Vespula vulgaris | Ves v 1 | phospholipase A1 | 35 | C | 105A |
| yellowjacket | Ves v 2 | hyaluronidase | 44 | P | 105A |
| | Ves v 5 | antigen 5 | 23 | C | 104 |
| Myrmecia pilosula | Myr p 1 | | | C | X70256 |
| Australian jumper ant | Myr p 2 | | | C | S81785 |
| Solenopsis geminata | Sol g 2 | | | | Hoffman p.c. |
| tropical fire ant | Sol g 4 | | | | Hoffman p.c. |
| Solenopsis invicta | Sol i 2 | | 13 | C | 110, 111 |
| fire ant | Sol i 3 | | 24 | C | 110 |
| | Sol i 4 | | 13 | C | 110 |
| Solenopsis saevissima | Sol s 2 | | | | Hoffman p.c. |
| Brazilian fire ant | | | | | |
| Triatoma protracta | Tria p 1 | Procalin | 20 | C | AF179004, 111A. |
| California kissing bug | | | | | |

H. Foods

| | | | | | |
|---|---|---|---|---|---|
| Gadus callarias | Gad c 1 | allergen M | 12 | C | 112, 113 |
| cod | | | | | |
| Salmo salar | Sal s 1 | parvalbumin | 12 | C | X97824 |
| Atlantic salmon | | | | | |
| Bos domesticus | Bos d 4 | alpha-lactalbumin | 14.2 | C | M18780 |
| domestic cattle | Bos d 5 | beta-lactoglobulin | 18.3 | C | X14712 |
| (milk) | Bos d 6 | serum albumin | 67 | C | M73993 |
| see also animals | Bos d 7 | immunoglobulin | 160 | | 77 |
| | Bos d 8 | caseins | 20-30 | | 77 |
| Gallus domesticus | Gal d 1 | ovomucoid | 28 | C | 114, 115 |
| chicken | Gal d 2 | ovalbumin | 44 | C | 114, 115 |
| | Gal d 3 | Ag22, conalbumin | 78 | C | 114, 115 |
| | Gal d 4 | lysozyme | 14 | C | 114, 115 |
| | Gal d 5 | serum albumin | 69 | C | X60688 |
| Metapenaeus ensis | Met e 1 | tropomyosin | | C | U08008 |
| shrimp | | | | | |
| Penaeus aztecus | Pen a 1 | tropomyosin | 36 | P | 116 |
| shrimp | | | | | |
| Penaeus indicus | Pen i 1 | tropomyosin | 34 | C | 116A |
| shrimp | | | | | |
| Penaeus monodon | Pen m 1 | tropomyosin | 38 | C | |
| black tiger shrimp | Pen m 2 | arginine kinase | 40 | C | AF479772, 117 |
| Todarodes pacificus | Tod p 1 | tropomyosin | 38 | P | 117A |
| squid | | | | | |
| Helix aspersa | Hel as 1 | tropomyosin | 36 | C | Y14855, 117B |
| brown garden snail | | | | | |
| Haliotis midae | Hal m 1 | | 49 | | 117C |
| abalone | | | | | |
| Rana esculenta | Ren e 1 | parvalbumin alpha | 11.9* | C | AJ315959 |
| edible frog | Ren e 2 | parvalbumin beta | 11.7* | C | AJ414730 |
| Brassica juncea | Bra j 1 | 2S albumin | 14 | C | 118 |
| oriental mustard | | | | | |
| Brassica napus | Bra n 1 | 2S albumin | 15 | P | 118A, P80208 |
| rapeseed | | | | | |
| Brassica rapa | Bra r 2 | hom: prohevein | 25 | | P81729 |
| turnip | | | | | |
| Hordeum vulgare | Hor v 15 | BMAI-1 | 15 | C | 119 |
| barley | Hor v 16 | alpha-amylase | | | |
| | Hor v 17 | beta-amylase | | | |
| | Hor v 21 | gamma-3 hordein | 34 | C | 119A, |
| | | | | | SW: P80198 |
| Secale cereale | Sec c 20 | secalin | | | see isoall. list |
| rye | | | | | |
| Triticum aestivum | Tri a 18 | agglutinin | | | |
| wheat | Tri a 19 | omega-5 gliadin | 65 | P | PIR: A59156 |
| Zea mays | Zea m 14 | lipid transfer prot. | 9 | P | P19656 |
| maise, corn | | | | | |
| Oryza sativa | Ory s 1 | | | C | 119B, U31771 |
| rice | | | | | |
| Apium gravaolens | Api g 1 | hom: Bet v 1 | 16* | C | Z48967 |
| celery | Api g 4 | profilin | | | AF129423 |
| | Api g 5 | | 55/58 | P | P81943 |
| Daucus carota | Dau c 1 | hom: Bet v 1 | 16 | C | 117D, see isoallergen |
| carrot | Dau c 4 | profilin | | C | AF456482 |
| list | | | | | |
| Corylus avellana | Cor a 1.04 | hom: Bet v 1 | 17 | C | see list of isoallergens |
| hazelnut | Cor a 2 | profilin | 14 | C | AF327622 |
| | Cor a 8 | lipid transfer protein | 9 | C | AF329829 |
| Malus domestica | Mal d 1 | hom: Bet v 1 | | C | see list of isoallergens |
| apple | Mal d 2 | hom: thaumatin | | C | AJ243427 |
| | Mal d 3 | lipid transfer protein | 9 | C | Pastorello p.c. |
| | Mal d 4 | profilin | 14.4* | C | sae list of isoallergens |
| Pyrus communis | Pyr c 1 | hom: Bet v 1 | 18 | C | AF05730 |
| pear | Pyr c 4 | profilin | 14 | C | AF129424 |
| | Pyr c 5 | hom: isoflavone reductas | 33.5 | C | AF071477 |
| Persea americana | Pers a 1 | endochitinase | 32 | C | Z78202 |
| avocado | | | | | |
| Prunus armeniaca | Pru ar 1 | hom: Bet v 1 | | C | U93165 |
| apricot | Pru ar 3 | lipid transfer protein | 9 | P | |
| Prunus avium | Pru av 1 | hom: Bet v 1 | | C | U66076 |
| sweet cherry | Pru av 2 | hom: thaumatin | | C | U32440 |
| | Pru av 3 | lipid transfer protein | 10 | C | AF221501 |
| | Pru av 4 | profilin | 15 | C | AF129425 |
| Prunus domestica | Pru d 3 | lipid transfer protein | 9 | P | 119C |
| European plum | | | | | |
| Prunus persica | Pru p 3 | lipid transfer protein | 10 | P | P81402 |
| peach | Pru p 4 | profilin | 14 | C | see isoallergen list |
| Asparagus officinalis | Aspa o 1 | lipid transfer protein | 9 | P | 119D |
| Asparagus | | | | | |
| Crocus sativus | Cro s 1 | | 21 | | Varasteh A-R p.c. |
| saffron crocus | | | | | |
| Lactuca sativa | Lac s 1 | lipid transfer protein | 9 | | Vieths p.c. |
| lettuce | | | | | |
| Vitis vinifera | Vit v 1 | lipid transfer protein | 9 | P | P80274 |
| grape | | | | | |
| Musa x paradisiaca | Mus xp 1 | profilin | 15 | C | AF377948 |
| banana | | | | | |
| Ananas comosus | Ana c 1 | profilin | 15 | C | AF377949 |
| pineapple | Ana c 2 | bromelain | 22.8* | C | 119E-G, D14059 |
| Citrus limon | Cit l 3 | lipid transfer protein | 9 | P | Torrejon p.c. |
| lemon | | | | | |
| Citrus sinensis | Cit s 1 | germin-like protein | 23 | P | Torrejon p.c. |
| sweet orange | Cit s 2 | profilin | 14 | P | Torrejon p.c. |
| | Cit s 3 | lipid transfer protein | 9 | P | Torrejon p.c. |
| Litchi chinensis | Lit c 1 | profilin | 15 | C | AY049013 |
| litchi | | | | | |
| Sinapis alba | Sin a 1 | 2S albumin | 14 | C | 120 |
| yellow mustard | | | | | |
| Glycine max | Gly m 1 | HPS | 7 | P | 120A |
| soybean | Gly m 2 | | 8 | P | A57106 |
| | Gly m 3 | profilin | 14 | C | see list of isoallergens |
| | Gly m 4 | (SAM22) PR-10 prot. | 17 | C | X60043, 120B |
| Vigna radiata | Vig r 1 | PR-10 protein | 15 | C | AY792956 |
| mung bean | | | | | |
| Arachis hypogaea | Ara h 1 | vicilin | 63.5 | C | L34402 |
| peanut | Ara h 2 | conglutin | 17 | C | L77197 |
| | Ara h 3 | glycinin | 60 | C | AF093541 |
| | Ara h 4 | glycinin | 37 | C | AF086821 |
| | Ara h 5 | profilin | 15 | C | AF059616 |
| | Ara h 6 | hom: conglutin | 15 | C | AF092846 |
| | Ara h 7 | hom: conglutin | 15 | C | AF091737 |
| | Ara h 8 | PR-10 protein | 17 | C | AY328088 |
| Lens culinaris | Len c 1 | vicilin | 47 | C | see list of isoallergens |
| lentil | Len c 2 | seed biotinylated prot. | 66 | P | 120C |
| Pisum savitum | Pis s 1 | vicilin | 44 | C | see list of isoallergens |
| pea | Pis s 2 | convicilin | 63 | C | pending |
| Actinidia chinensis | Act c 1 | cysteine protease | 30 | P | P00785 |
| kiwi | Act c 2 | thaumatin-like protein | 24 | P | SW: P81370, 121 |
| Capsicum annuum | Cap a 1w | osmotin-like protein | 23 | C | AJ297410 |
| bell pepper | Cap a 2 | profilin | 14 | C | AJ417552 |
| Lycopersicon esculentum | Lyc e 1 | profilin | 14 | C | AJ417553 |
| tomato | Lyc e 2 | b-fructofuranosidase | 50 | C | see isoallergen list |
| | Lyc e 3 | lipid transfer prot. | 6 | C | U81996 |
| Solanum tuberosum | Sola t 1 | patatin | 43 | P | P15476 |
| potato | Sola t 2 | cathepsin D inhibitor | 21 | P | P16348 |
| | Sola t 3 | cysteine protease inhibitor | 21 | P | P20347 |
| | Sola t 4 | aspartic protease inhibitor | 16 + 4 | P | P30941 |
| Bertholletia excelsa | Ber e 1 | 2S albumin | 9 | C | P04403, M17146 |
| Brazil nut | Ber e 2 | 11S globulin seed storage protein | 29 | C | AY221641 |
| Juglans nigra | Jug n 1 | 2S albumin | 19* | C | AY102930 |
| black walnut | Jug n 2 | vicilin-like prot. | 56* | C | AY102931 |
| Juglans regia | Jug r 1 | 2S albumin | | C | U66866 |
| English walnut | Jug r 2 | vicilin | 44 | C | AF066055 |
| | Jug r 3 | lipid transfer protein | 9 | P | Pastorello |
| Anacardium occidentale | Ana o 1 | vicilin-like protein | 50 | C | see isoallergen list |
| Cashew | Ana o 2 | legumin-like protein | 55 | C | AF453947 |
| | Ana o 3 | 2S albumin | 14 | C | AY081853 |
| Ricinus communis | Ric c 1 | 2S albumin | | C | P01089 |
| Castor bean | | | | | |
| Sesamum indicum | Ses i 1 | 2S albumin | 9 | C | 121A, AF240005 |
| sesame | Ses i 2 | 2S albumin | 7 | C | AF091841 |
| | Ses i 3 | 7S vicilin-like globulin | 45 | C | AF240006 |
| | Ses i 4 | oleosin | 17 | C | AAG23840 |
| | Ses i 5 | oleosin | 15 | C | AAD42942 |
| Cucumis melo | Cuc m 1 | serine protease | 66 | C | D32206 |
| muskmelon | Cuc m 2 | profilin | 14 | C | AY271295 |
| | Cuc m 3 | pathogenesis-rel p. PR-1 | 16* | P | P83834 |

I. Others

| | | | | | |
|---|---|---|---|---|---|
| Anisakis simplex | Ani s 1 | | 24 | P | 121B, A59069 |
| nematode | Ani s 2 | paramyosin | 97 | C | AF173004 |
| | Ani s 3 | tropomyosin | 41 | C | 121C, Y19221 |
| | Ani s 4 | | 9 | P | P83885 |
| Argas reflexus | Arg r 1 | | 17 | C | AJ697694 |
| pigeon tick | | | | | |
| Ascaris suum | Asc s 1 | | 10 | P | 122 |
| worm | | | | | |
| Carica papaya | Car p 3w | papain | 23.4* | C | 122A, M15203 |
| papaya | | | | | |
| Dendronephthya nipponica | Den n 1 | | 53 | P | 122B |
| soft coral | | | | | |
| Hevea brasiliensis | Hev b 1 | elongation factor | 58 | P | 123, 124 |
| rubber (latex) | Hev b 2 | 1,3-glucanase | 34/36 | C | 125 |
| | Hev b 3 | | 24 | P | 126, 127 |
| | Hev b 4 | component of | 100-115 | P | 128 |
| | | microhelix complex | | | |
| | Hev b 5 | | 16 | C | U42640 |
| | Hev b 6.01 | hevein precursor | 20 | C | M36986, p02877 |
| | Hev b 6.02 | hevein | 5 | C | M36986, p02877 |
| | Hev b 6.03 | C-terminal fragment | 14 | C | M36986, p02877 |
| | Hev b 7.01 | hom: patatin from B-serum | 42 | C | U80598 |
| | Hev b 7.02 | hom: patatin from C-serum | 44 | C | AJ223038 |
| | Hev b 8 | profilin | 14 | C | see list of isoallergens |
| | Hev b 9 | enolase | 51 | C | AJ132580 |
| | Hev b 10 | Mn superoxide dismut. | 26 | C | see list of isoallergens |
| | Hev b 11 | class 1 chitinase | | C | see list of isoallergens |
| | Hev b 12 | lipid transfer protein | 9.3 | C | AY057860 |
| | Hev b 13 | esterase | 42 | P | P83269 |
| Homo sapiens | Hom s 1 | | 73* | C | Y14314 |
| human autoallergens | Hom s 2 | | 10.3* | C | X80909 |
| | Hom s 3 | | 20.1* | C | X89985 |
| | Hom s 4 | | 36* | C | Y17711 |
| | Hom s 5 | | 42.6* | C | P02538 |
| Triplochiton scleroxylon | Trip s 1 | class 1 chitinase | 38.5 | P | Kespohl p.c. |
| obeche | | | | | |

### REFERENCES

1 Marsh, D. G., and L. R. Freidhoff. 1992. ALBE, an allergen database. IUIS, Baltimore, Md., Edition 1.0.
2 Marsh, D. G. et al. 1986. Allergen nomenclature. Bull WHO 64:767-770.
3 King, T. P. et al. 1964. Biochemistry 3:458-468.
4 Lowenstein, H. 1980. Allergy 35:188-191.
5 Aukrust, L. 1980. Allergy 35:206-207.
6 Demerec, M. et al. 1966. Genetics 54:61-75.
7 Bodmer, J. G. et al. 1991. Immunogenetics 33:301-309.
8 Griffith, I. J. et al. 1991. Int. Arch. Allergy Appl. Immunol. 96:296-304.
9 Roebber, M. et al. 1985. J. Immunol. 134:3062-3069.
10 Metzler, W. J. et al. 1992. Biochemistry 31:5117-5127.
11 Metzler, W. J. et al. 1992. Biochemistry 31:8697-8705.
12 Goodfriend, L. et al. 1979. Fed. Proc. 38:1415.
13 Ekramoddoullah, A. K. M. et al. 1982. Mol. Immunol. 19:1527-1534.
14 Ansari, A. A. et al. 1987. J. Allergy Clin. Immunol. 80:229-235.
15 Morgenstern, J. P. et al. 1991. Proc. Natl. Acad. Sci. USA 88:9690-9694.
16 Griffith, I. J. et al. 1992. Gene 113:263-268.
17 Weber, A. et al. 1986. Biochem. Physiol. 83B: 321-324.
18 Weber, A. et al. 1987. Allergy 42:464-470.
19 Stanworth, D. R. et al. 1990. Bulletin WHO 68:109-111.
20 Rafnar, T. et al. 1991. J. Biol. Chem. 266: 1229-1236.
21 Rogers, B. L. et al. 1991. J. Immunol. 147:2547-2552.
22 Klapper, D. G. et al. 1980. Biochemistry 19:5729-5734.
23 Ghosh, B. et al. 1993. J. Immunol. 150:5391-5399.
24 Roebber, M. et al. 1983. J. Immunol. 131:706-711.
25 Lubahn, B., and D. G. Klapper. 1993. J. Allergy Clin. Immunol. 91:338.
26 Roebber, M., and D. G. Marsh. 1991. J. Allergy Clin. Immunol. 87:324.
27 Goodfriend L. et al. Mol Immunol 22: 899-906, 1985.
28 Himly M. et al. FASEB J 17: 106-108, 2003.
28A Nilsen, B. M. et al. 1991. J. Biol. Chem. 266:2660-2668.
29 Wopfner N. et al. Biol Chem 383: 1779-1789, 2002.
29A Jimenez A. et al. 1994. Int Arch Allergy Immunol 105:297-307.
29B Barderas R. et al. Int Arch Allergy Immunol 127: 47-54, 2002.
29C Carnés J. et al. Allergy 56, Supplement 68: 274, 2001.
29D Giuliani A. et al. Allergy 42: 434-440, 1987.
30 Smith, P. M. et al. 1996. J. Allergy Clin. Immunol. 98:331-343.
31 Suphioglu, C. et al. 1997. FEBS Lett. 402:167-172.
31a Asturias J. A. et al. 1997. Clin Exp Allergy 27:1307-1313.
32 Mecheri, S. et al. 1985. Allergy Appl. Immunol. 78:283-289.
33 Roberts, A. M. et al. 1993. Allergy 48:615-623.
33a Guerin-Marchand, C. et al. 1996. Mol. Immunol. 33:797-806.
34 Klysner, S. et al. 1992. Clin. Exp. Allergy 22: 491-497.
35 Perez, M. et al. 1990. J. Biol. Chem. 265:16210-16215.
36 Griffith, I. J. et al. 1991. FEBS Letters 279:210-215.
37 Ansari, A. A. et al. 1989. J. Biol. Chem. 264:11181-11185.
37a Sidoli, A. et al. 1993. J. Biol. Chem. 268:21819-21825.
38 Ansari, A. A. et al. 1989. Biochemistry 28:8665-8670.
39 Singh, M. B. et al. 1991. Proc. Natl. Acad. Sci. 88:1384-1388.
39a van Ree R. et al. 1995. J Allergy Clin Immunol 95:970-978.
40 Suphioglu, C. and Singh, M. B. 1995. Clin. Exp. Allergy 25:853-865.
41 Dolecek, C. et al. 1993. FEBS Lett. 335:299-304.
41A Fischer S. et al. 1996. J Allergy Clin Immunol 98:189-198.
42 Matthiesen, F., and H. Lowenstein. 1991. Clin. Exp. Allergy 21:297-307.
43 Petersen, A. et al. 1995. Int. Arch. Allergy Immunol. 108:55-59.
43A Marknell DeWitt A. et al. Clin Exp Allergy 32: 1329-1340, 2002.
44 Valenta, R. et al. 1994. Biochem. Biophys. Res. Commun. 199:106-118.
46 Esch, R. E., and D. G. Klapper. 1989. Mol. Immunol. 26:557-561.
47 Olsen, E. et al. 1991. J. Immunol. 147:205-211.
48 Avjioglu, A. et al. 1993. J. Allergy Clin. Immunol. 91:340.
52 Kos T. et al. 1993. Biochem Biophys Res Commun 196:1086-92.
53 Díaz-Perales A. et al. 2000. Clin Exp Allergy 30:1403-1410.
54 Ipsen, H., and O. C. Hansen. 1991. Mol. Immunol. 28: 1279-1288.
55 Taniai, M. et al. 1988. FEBS Lett. 239:329-332.
56 Griffith, I. J. et al. 1993. J. Allergy Clin. Immunol. 91:339.
57 Sakaguchi, M. et al. Allergy 45: 309-312, 1990.
57A Yokoyama M. et al. Biochem Biophys Res Commun 275: 195-202, 2000.
57B Midoro-Horiuti T. et al. J Immunol 164: 2188-2192, 2000.
57C Tinghino R. et al. J. Allergy Clin. Immunol. 101: 772-777, 1998.
58 Gross G N et al. Scand J Immunol 8: 437-441, 1978.
58A Obispo T M et al. Clin Exp Allergy 23: 311-316, 1993.
58B Midoro-Horiuti T. et al. Clin Exp Allergy 31: 771-778, 2001.
59 Lombardero M. et al. Clin. Exp. Allergy 24: 765-770, 1994.
60 Villalba, M. et al. Eur. J. Biochem. 216: 863-869, 1993.
60A Asturias J A et al. J Allergy Clin Immunol 100: 365-372, 1997.
60B Batanero E. et al. Eur J Biochem 241: 772-778, 1996.
60C Batanero E. et al. FEBS Lett. 410: 293-296, 1997.
60D Tejera M L et al. J Allergy Clin Immunol 104: 797-802, 1999.
60E Ledesma A. et al. FEBS Lett 466: 192-196, 2000.
60F Barral P. et al. J Immunol 172: 3644-3651, 2004.
61 Yi F C et al. Clin Exp Allergy 32: 1203-1210, 2002.
61A Ramos J D et al. Int Arch Allergy Immunol 126: 286-293, 2001.
62 Chua, K. Y. et al. J. Exp. Med. 167: 175-182, 1988.
62A Chua, K. Y. et al. Int. Arch. Allergy Appl. Immunol. 91: 118-123, 1990.
62B Smith A M et al. Int Arch Allergy Immunol 124: 61-63, 2001.
b 62C Smith A M et al. J Allergy Clin Immunol 107: 977-984, 2001.
63 Smith W A, Thomas W R. Int Arch Allergy Immunol 109: 133-140, 1996.
64 Lake, F. R. et al. J. Allergy Clin. Immunol. 87: 1035-1042, 1991.
65 Tovey, E. R. et al. J. Exp. Med. 170: 1457-1462, 1989.
66 Yasueda, H., T. Shida, T. Ando, S. Sugiyama, and H. Yamakawa. 1991. Allergenic and proteolytic properties of fourth allergens from Dermatophagoides mites. In: “Dust Mite Allergens and Asthma. Report of the 2nd international workshop” A. Todt, Ed., UCB Institute of Allergy, Brussels, Belgium, pp. 63-64.
67 Shen, H.-D. et al. Clin. Exp. Allergy 23: 934-940, 1993.
67A O'Neil G M et al. Biochim Biophys Acta, 1219: 521-528, 1994.
67B King C. et al. J Allergy Clin Immunol 98: 739-747, 1996.
68 Lind P. et al. J. Immunol. 140: 4256-4262, 1988.
69 Dilworth, R. J. et al. Clin. Exp. Allergy 21: 25-32, 1991.
70 Nishiyama, C. et al. Int. Arch. Allergy Immunol. 101: 159-166, 1993.
70A Trudinger, M. et al. Clin. Exp. Allergy 21: 33-38, 1991.
71 Shen H D et al. Clin Exp Allergy 25: 1000-1006, 1995.
71A Tategaki A. et al. ACI International suppl. 1: 74-76, 2000.
72 Aki T. et al. J Allergy Clin Immunol 96: 74-83, 1995.
72A Tsai L. et al. Clin Exp Allergy 29: 1606-1613, 1999.
72B Gafvelin G. et al. J Allergy Clin Immunol 107: 511-518, 2001.
73 van Hage-Hamsten. et al. J. Allergy Clin. Immunol. 91:353, 1993.
74 Varela J. et al. Eur J Biochem 225: 93-98, 1994.
74A Schmidt M. et al. FEBS Lett 370: 11-14, 1995.
75 Eriksson T L J et al. Eur. J. Biochem. 268: 287-294, 2001.
75A Saarne T. et al. Int Arch Allergy Immunol 130: 258-265, 2003.
75B Eriksson T L et al. Eur. J. Biochem. 251 (1-2), 443-447, 1998.
76 Rautiainen J, Rytkonen M, Pelkonen J, Pentikainen J, Perola O, Virtanen T, Zeiler T, Mantyjarvi R. BDA20, a major bovine dander allergen characterised at the sequence level is Bos d 2. Submitted.
77 Gjesing B, Lowenstein H. Ann Allergy 53:602, 1984.
78 de Groot, H. et al. J. Allergy Clin. Immunol. 87:1056-1065, 1991.
79 Konieczny, A. Personal communication; Immunologic Pharmaceutical Corp.
79A Bulone, V. Eur J Biochem 253: 202-211, 1998.
79B Swiss-Prot acc. P81216, P81217.
79C Dandeu J. P. et al. (1993). J. Chromatogr. 621:23-31.
79D Goubran Botros H. et al. 1998. J. Chromatogr. B 710:57-65.
79E Hilger C. et al. Allergy 52: 179-187; and Hilger C. et al. Gene 169:295-296, 1996.
79F Ichikawa K. et al. Clin Exp Allergy, In Press 2001.
80 Fahlbusch B. et al. Allergy 57: 417-422, 2002.
81 McDonald, B. et al. 1988. J. Allergy Clin. Immunol. 83:251.
81A Clarke, A. J. et al. 1984. EMBO J 3:1045-1052.
82 Longbottom, J. L. 1983. Characterisation of allergens from the urines of experimental animals. McMillan Press, London, pp. 525-529.
83 Laperche, Y. et al. 1983. Cell 32:453-460.
83A Bush R K et al. 1999. J Allergy Clin Immunol 104:665-671.
83B Aukrust L, Borch S M. 1979. Int Arch Allergy Appl Immunol 60:68-79.
83C Sward-Nordmo M. et al. 1988. Int Arch Allergy Appl Immunol 85:288-294.
84 Shen, et al. J. Allergy Clin. Immunol. 103:S157, 1999.
84A Crameri R. Epidemiology and molecular basis of the involvement of Aspergillus fumigatus in allergic diseases. Contrib. Microbiol. Vol. 2, Karger, Basel (in press).
84B Shen, et al. (manuscript submitted), 1999
84C Shen H D et al. Vacuolar serine proteinase: A major allergen of Aspergillus fumigatus. 10th International Congress of Immunology, Abstract, 1998.
85 Kumar A. et al. 1993. J. Allergy Clin. Immunol. 91:1024-1030.
85A Saxena S. et al. 2003. Clin Exp Immunol 134:86-91.
85B Baur X. et al. Allergy 57: 943-945, 2002.
86A Shen H D et al. 1996. Clin Exp Allergy 26:444-451.
86B Shen, et al. Abstract; The XVIII Congress of the European Academy of Allergology and Clinical Immunology, Brussels, Belgium, 3-7 Jul. 1999.
87 Shen H D et al. Clin Exp Allergy 29: 642-651, 1999.
87A Shen H D et al. Clin Exp Allergy 25: 350-356, 1995.
87B Shen H D et al. J Lab Clin Med 137: 115-124, 2001.
88 Woodfolk J A et al. 1998. J Biol Chem 273:29489-96.
88A Deuell, B. et al. 1991. J. Immunol. 147:96-101.
89 Shen, H. D. et al. 1991. Clin. Exp. Allergy 21:675-681.
89A Horner W E et al. 1995. Int Arch Allergy Immunol 107:298-300.
89B Chang C Y et al. J Biomed Sci 9: 645-655, 2002.
90 Yasueda H. et al. Biochem Biophys Res Commun 248: 240-244, 1998. NB:strain TIMM2782 (Teikyo University Institute for Medical Mycology) equal to strain CBS1878 (Central Bureau von Schimmelkulturen).
90A Onishi Y. et al. Eur J Biochem 261: 148-154, 1999. NB: strain TIMM2782 (Teikyo University Institute for Medical Mycology) equal to strain CBS1878 (Central Bureau von Schimmelkulturen).
91 Schmidt M. et al. Eur J Biochem 246:181-185, 1997. NB: strain ATCC no. 42132 (American Type Culture Collection).
91A Rasool O. et al. Eur J Biochem 267: 4355-4361, 2000. NB: strain ATCC no. 42132 (American Type Culture Collection).
91B NB: strain 4625 (Indian Agricultural Research Institute, PUSA; New Delhi, India).
92 Kuchler, K. et al. 1989. Eur. J. Biochem. 184:249-254.
93 Gmachl, M., and G. Kreil. 1993. Proc. Natl. Acad. Sci. USA 90:3569-3573.
93A Hoffman D R. 1977. J Allergy Clin. Immunol. 59:364-366.
94 Habermann, E. 1972. Science 177:314-322.
95 Hoffman D R, Jacobson R S. 1996. J. Allergy Clin. Immunol. 97:812-821.
95A Hoffman D R, El-Choufani A E, Smith M M, de Groot H. 2001. Occupational allergy to bumblebee venom: Allergens of Bombus terrestris. J Allergy Clin Immunol In press.
95B Helm R. et al. 1996. J Allerg Clin Immunol 98:172-180.
95C Pomes A. et al. 1998. J Biol Chem 273:30801-30807.
96 Arruda L K et al. J Biol Chem 270:19563-19568, 1995.
97 Arruda L K et al. J Biol Chem 270:31196-31201, 1995.
98 Arruda L K et al. Int Arch Allergy Immunol 107:295-297, 1995.
98A Wu C H et al. 1998. J Allergy Clin Immunol 101:832-840.
98B Melen E. et al. 1999. J Allergy Clin Immunol 103:859-64.
98C Wu C H et al. J Biol Chem 271:17937-17943, 1996.
98D Wu C H et al. Molecular Immunol 34:1-8, 1997.
98E Santos A B R et al. 1999. J Allergy Clin Immunol 104:329-337.
98F Asturias J A et al. 1999. J Immunol 162:4342-4348.
99 Mazur, G. et al. 1990. Monog. Allergy 28:121-137.
99A Moneo I. et al. Allergy 58: 34-37, 2003.
100 Soldatova, L. et al. 1993. FEBS Letters 320:145-149.
101 Lu, G. et al. 1994. J. Allergy Clin. Immunol. 93:224.
102 Fang, K. S. F. et al. 1988. Proc. Natl. Acad. Sci., USA 85:895-899.
103 King, T. P. et al. 1990. Prot. Seq. Data Anal. 3:263-266.
104 Lu, G. et al. 1993. J. Immunol. 150: 2823-2830.
105 King, T. P. and Lu, G. 1997. Unpublished data.
105A King T P et al. 1996. J. Allergy Clin. Immunol. 98:588-600.
106 Hoffman, D. R. 1993. J. Allergy Clin. Immunol. 92:707-716.
107 Hoffman D R. 1992. Unpublished data.
108 Hoffman D R. J. Allergy Clin. Immunol. 91:187, 1993.
109 Jacobson R S et al. J. Allergy Clin. Immunol. 89:292, 1992.
110 Hoffman D R. J. Allergy Clin. Immunol 91: 71-78, 1993.
111 Schmidt M. et al. FEBS Letters 319: 138-140, 1993.
111A Paddock C D et al. J Immunol 167: 2694-2699, 2001.
112 Elsayed S, Bennich H. Scand J Immunol 3: 683-686, 1974.
113 Elsayed S. et al. Immunochemistry 9: 647-661, 1972.
114 Hoffman, D. R. 1983. J. Allergy Clin. Immunol. 71: 481-486.
115 Langeland, T. 1983. Allergy 38:493-500.
116 Daul C B, Slattery M, Morgan J E, Lehrer S B. 1993. Common crustacea allergens: identification of B cell epitopes with the shrimp specific monoclonal antibodies. In: “Molecular Biology and Immunology of Allergens” (D. Kraft and A. Sehon, eds.). CRC Press, Boca Raton. pp. 291-293.
116A Shanti K N et al. J. Immunol. 151: 5354-5363, 1993.
117 Yu C J et al. J Immunol 170: 445-453, 2003.
117A Miyazawa M et al. J. Allergy Clin. Immunol. 98: 948-953, 1996.
117B Asturias J A et al. Int Arch Allergy Immunol 128: 90-96, 2002.
117C Lopata A L et al. J. Allergy Clin. Immunol. 100: 642-648, 1997.
117D Hoffmann-Sommergruber K. et al. Clin. Exp. Allergy 29: 840-847, 1999.
118 Monsalve R I et al. Biochem. J. 293: 625-632 1993.
118A. Monsalve R I et al. 1997. Clin Exp Allergy 27:833-841.
119 Mena, M. et al. Plant Molec. Biol. 20: 451-458, 1992.
119A Palosuo K. et al. J. Allergy Clin. Immunol. 108: 634-638, 2001.
119B Xu H. et al. Gene 164: 255-259, 1995.
119C Pastorello E A et al. J. Allergy Clin. Immunol. 94: 699-707, 1994.
119D Diaz-Perales A. et al. J Allergy Clin Immunol 110: 790-796, 2002.
119E Galleguillos F. Rodriguez J C. Clin Allergy 8: 21-24, 1978.
119F Baur X. Clin Allergy 9: 451-457, 1979.
119G Gailhofer G. et al. Clin Allergy 18: 445-450, 1988.
120 Menendez-Arias, L. et al. 1988. Eur. J. Biochem. 177:159-166.
120A Gonzalez R. et al. Lancet 346:48-49, 1995.
120B Kleine-Tebbe J. et al. J Allergy Clin Immunol 110: 797-804, 2002.
120C Sanchez-Monge R. et al. J. Allergy Clin. Immunol. 106: 955-961, 2000.
121 Gavrovic-Jankulovic M. et al. J Allergy Clin Immunol 110: 805-810, 2002.
121A Pastorello E A et al. J. Chromatogr. B Biomed. Sci. Appl. 756: 85-93, 2001.
121B Moneo I. et al. J. Allergy Clin. Immunol. 106: 177-182, 2000.
121C Asturias J A et al. 2000. Allergy 55:898-890.
122 Christie, J. F. et al. 1990. Immunology 69:596-602.
122A Baur X. et al. Clin Allergy 12: 9-17, 1982.
122B Onisuka R. et al. Int Arch Allergy Immunol 125: 135-143, 2001.
123 Czuppon A B et al. J Allergy Clin Immunol 92:690-697, 1993.
124 Attanayaka D P S T G et al. 1991. Plant Mol Biol 16:1079-1081.
125 Chye M L, Cheung K Y. 1995. Plant Mol Biol 26:397-402.
126 Alenius H. et al. 1993. Int Arch Allergy Immunol 102:61-66.
127 Yeang H Y, Cheong K F, Sunderasan E, Hamzah S, Chew N P, Hamid S, Hamilton R G, Cardosa M J. 1996. The 14.6 kD (REF, Hev b 1) and 24 kD (Hev b 3) rubber particle proteins are recognised by IgE from Spina Bifida patients with Latex allergy. J Allerg Clin Immunol in press.
128 Sunderasan E. et al. 1995. J nat Rubb Res 10:82-99.

The knowledge of the nucleic acid sequences encoding these allergens allows their recombinant production. Therefore especially these allergens are preferably used in immunotherapies and in methods according to the present invention.

Another aspect of the present invention relates to a method for evaluating the allergen sensitivity of an individual and/or the clinical efficacy of an allergen immunotherapy comprising the steps:
providing cells capable of releasing mediators in response to an IgE-allergen complex,
contacting said cells with serum and/or plasma of said individual spiked with at least one pure allergen or derivative thereof, and
determining the amounts of mediators released from said sample and evaluating the allergen sensitivity of the individual prior to therapy and/or the clinical efficacy of the immunotherapy by comparing said amounts.

The cells which are capable of releasing mediators comprise normally IgE molecules bound thereto. Such cells can be isolated from samples which are obtained from the individual subjected to the method according to the present invention or from other individuals. Of course, it is also possible to use cell lines capable of binding IgE in a method according to the present invention.

The method according to the present invention is especially suited for the determination of the allergen sensitivity of an individual because it allows to determine the ratio between the allergen specific IgE and IgG molecules in the plasma and serum of said individual. Since only IgE-allergen complexes and not free IgE are able to induce the release of mediators from mediator-releasing cells like leukozytes the level of released mediator correlates with the amount of IgE-complex present in the sample. In turn the amount of IgE-complex in said sample correlates with the amount of allergen specific IgE, allergen and allergen specific antibodies other than IgE such as IgG, IgA or IgM which compete with IgE for the free allergen and consequently inhibits the formation of an IgE-allergen complex. This means that a low level of allergen specific IgE or a high level of allergen specific IgG leads to the formation of a low number of IgE complex and thus to a reduced mediator release.

The concentration of allergen in said serum and/or plasma is preferably within 1 ng/ml to 100 μg/ml, more preferably within 1 pg/ml to 10 μg/ml.

Another aspect of the present invention relates to a kit for evaluating the allergen sensitivity of an individual and/or the clinical efficacy of an allergen immunotherapy for at least one allergy comprising
at least one allergen for inducing a mediator release of cells capable of releasing the mediator in response to an allergen,
means for detecting mediator, and
optionally at least one mediator standard

The kit provided herein comprises at least one allergen, which can be used to induce the release of a mediator from mediator releasing cells contained in a sample. The released mediator is then detected directly or preferably—after the removal of solid parts of the sample—in the supernatant of the reaction mixture. Optionally also means for the detection of IgE molecules binding said allergen are enclosed in the kit according to the present invention. IgE is able to bind a distinct allergen and to mediate, when bound to a mediator releasing cell and the allergen, the release of mediator from said cells. However, IgE specific for an allergen is not normally detected in the blood and is only produced when a person becomes sensitised to an allergen. In order to accurately determine the amount of mediator in the sample (for the provision of a standard curve) a mediator standard may be optionally part of the kit.

Preferably the cells are mast and/or basophilic and/or eosinophilic cells.

According to another preferred embodiment of the present invention the allergen is selected from the group consisting of major birch pollen allergens, in particular Bet v 1 and Bet v 4, major timothy grass pollen allergens, in particular Phl p 1, Phl p 2, Phl p 5, Phl p 6 and Phl p 7, major house dust mite allergens, in particular Der p 1 and Der p 2, major cat allergen Fel d 1, major bee allergens, major wasp allergens, profilins, especially Phl p 12, and storage mite allergens, especially Lep d 2 and the allergens listed in table 1.

The means for detecting mediators are preferably antibodies.

A mediator, as outlined above, is preferably detected by immunological methods. Therefore the kit may provide at least one antibody which is able to bind specifically mediator. Preferably enzyme linked immuno sorbent assays (ELISA), radio immuno assays (RIA) or lateral flow devices are employed.

Another aspect of the present invention relates to a kit for evaluating the allergen sensitivity of an individual or the clinical efficiency of an allergen immunotherapy for at least one allergy comprising at least two of the following components:
at least one allergen for inducing a mediator release of cells capable of releasing mediators in response to an allergen,
means for detecting the mediator,
at least one mediator standard, and
cells capable of releasing mediators in response to an IgE-allergen complex.

The present invention is further illustrated by the following figures and example, without being restricted thereto.

FIG. 1 shows the association of results from intradermal end-point titration (x-axis: Allergen concentration giving the first positive reaction) and rBet v 1-specific serum IgE (y-axis: kU/L CAP System).

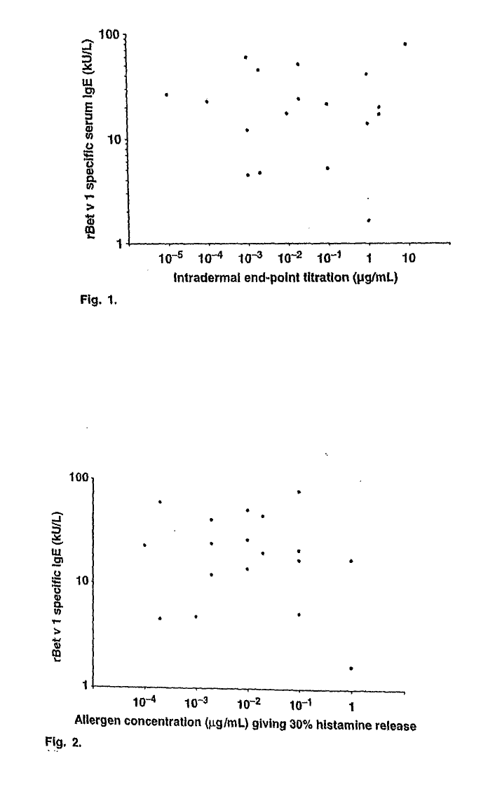

FIG. 2 shows the association of results from basophil histamine release (x-axis: Allergen concentration giving 30% histamine release) and rBet v 1-specific serum IgE (y-axis: kU/L CAP System).

FIG. 3 shows the association of results from intradermal end-point titration (x-axis: Allergen concentration giving the first positive reaction) and results from basophil histamine release (y-axis: Allergen concentration giving 30% histamine release).

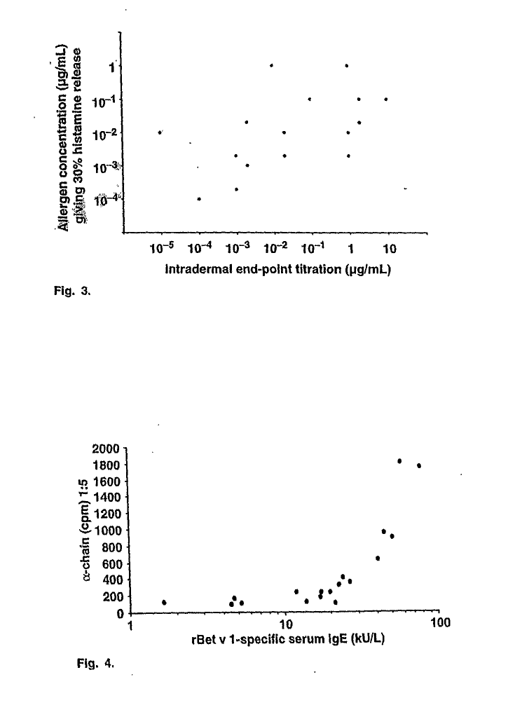

FIG. 4 shows the association of Bet v 1-specific IgE determined by CAP (x-axis: kU/L) and of rBet v 1-specific IgE determined with labelled a-chain (y-axis: counts per minute (c.p.m.); 1:5 serum dilution).

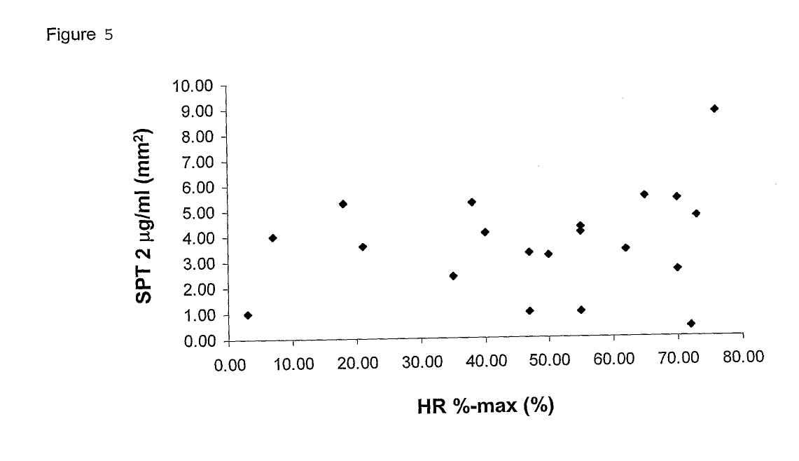

FIG. 5 shows the association of results from basophil histamine release (x-axis: maximal histamine release (%)) and results from skin prick testing (y-axis: weal reaction (mm²) induced by skin prick testing with 2 μg/ml of recombinant Bet v 1).

### EXAMPLES

### Example 1

The cross-linking of effector cell (mast cells and basophils)-bound IgE antibodies by allergens is a crucial event for the induction of the immediate symptoms of type I allergy (Kawakami T, et al., Nat Rev Immunol (2002) 2:773-86). As described in the classical experiments by Prausnitz and Küstner (Prausnitz C, at al., Centralbe F Bact 1 Abt Orig (1921) 86:160-8), this event depends on three major factors, i.e. allergen-specific IgE antibodies, effector cells and allergens. Because the characterisation of IgE antibodies and the development of diagnostic tests capable of measuring the precise amount of allergen-specific IgE antibodies, several studies have investigated the association of allergen-specific serum IgE levels and biological sensitivity to allergens in allergic patients (Stenius B, et al., Clin Allergy (1971) 1:37-55; Bryant D H, et al., Clin Allergy (1975) 5:145-57; Pauli G, et al., Clin Allergy (1977) 7:337-46; Bousquet J, et al., Clin Allergy (1987) 17:529-36; Witteman A M, et al., J Allergy Clin Immunol (1996) 97:16-25; Niederberger V, et al. J Invest Dermatol (2001) 117:848-51; Norman P S, et al., J Allergy Clin Immunol (1973) 52:210-24; Lichtenstein L M, et al. J Allergy Clin Immunol (1971) 47:103 (A37)). It is well established that the presence of allergen-specific serum IgE is a pre-requisite for the occurrence of an immediate type of reaction, but whether the amount of allergen-specific IgE correlates with immediate type sensitivity to the given allergen has been a matter of great debate. To address the problem almost all of the investigations carried out in the past have used allergen extracts, i.e. mixtures of allergens and non-allergenic molecules (Stenius B, et al., Clin Allergy (1971) 1:37-55; Bousquet J, et al., Clin Allergy (1987) 17:529-36; Norman P S, et al., J Allergy Clin Immunol (1973) 52:210-24; Lichtenstein L M, et al. J Allergy Clin Immunol (1971) 47:103 (A37)). This is the reason why these studies could not analyse the association between allergen-specific IgE levels and biological activities at molecular levels. Recent studies using purified natural and recombinant allergens to re-investigate the relation between skin sensitivity and allergen-specific IgE levels report considerable discrepancies between these parameters (Witteman A M, et al., J Allergy Clin Immunol (1996) 97:16-25; Niederberger V, et al. J Invest Dermatol (2001) 117:848-51). In this example, purified recombinant Bet v 1, the major birch pollen allergen, was used as a paradigmatic tool to further investigate the association between allergen-specific IgE levels, effector cell responses and in vivo sensitivity. In a population of 18 birch pollen-allergic patients, selected on well-defined clinical criteria, and out of the pollen season, skin sensitivity and basophil degranulation in response to defined amounts of structurally folded recombinant Bet v 1 was quantified. The results of the biological and of the serological tests were compared. For the measurement of Bet v 1-specific IgE antibody levels two different assays were used: one to detect any Bet v 1-specific IgE, and the other to detect Bet v 1-specific IgE able to bind to effector cells.

Material and Methods

Study Population

The examination of the patients was performed between January and April before the beginning of the birch pollen season. Eighteen patients, eight women and 10 men aged between 28 and 58 years (mean age: 45.6 years), were included in the study on the basis of clinical history of birch pollinosis and positive skin prick tests to birch pollen extract. All patients had moderate to severe rhino-conjunctivitis first diagnosed at least 3 years before. Five patients had mild asthma during birch pollen season and 12 patients had oral allergy syndrome with fruits of the Rosaceae family (apple, peach, apricot and almonds) and vegetables from Solanaceae (potato, tomato) and Apiaceae family (celery, carrot). Skin prick tests with a standard panel of respiratory allergens (Stallergènes, France) consisting of house dust mites, mixtures of fungal allergens, dog and cat dander, cockroach, grass, trees (including birch, olive and ash) and weed pollens were performed to identify the sensitisation profile. Patients' characteristics are displayed in the following Table 1.

Study Design

To analyse the possible association between allergen-specific IgE levels, skin sensitivity, and basophil degranulation, patients were bled and their skin was tested on the same day. The analyses were carried out strictly out of the birch pollen season to exclude effects because of seasonal allergen contact. Patients were not allowed to take anti-allergic or anti-inflammatory medication at least 1 week before the study was performed. None of the patients had received allergenspecific immunotherapy over the last 5 years. After informed consent was given, blood was collected for basophil histamine release and for serum sampling. Immediately thereafter, intradermal skin tests were performed using the end-point titration method (Grammer L C, et al., J Allergy Clin Immunol (1985) 76:123-7).

Detection and Quantification of Allergen-Specific Antibodies

Allergen-specific IgG1 to IgG4 subclass levels as well as allergen-specific IgM and IgA levels were measured by ELISA using isotype-specific monoclonal antibodies as described (Vrtala S, et al., J Allergy Clin Immunol (1996) 97:781-7). Results represent means of duplicate determinations and are shown as OD values corresponding to the amount of bound antibodies.

Basophil Histamine Release Test

The challenge of whole blood with rBet v 1 and anti-IgE as a positive control was performed in a dose response fashion according to the method described by Tanisaki et al. (Tanisaki Y, et al., Int Arch Allergy Appl Immunol (1984) 73:141-5). Ten millilitres of venous blood was drawn into a plastic syringe containing 1 ml of heparin. 250 μl of different concentrations of rBet v 1 (from 10⁻⁴ to 10 mg/ml) or anti-IgE (from 10⁻⁴ to 10⁻³; e-specific, Dako, Glostrup, Denmark) were added to the test tubes containing 500 ml of whole blood diluted 1:4 in Tris buffer (10 mmol/l Tris, 136 mmol/l NaCl, 2.7 mmol/l KCl, 0.23 mmol/l MgCl₂, 1.8 mmol/L CaCl₂, 5.5 mmol/l glucose; pH 7.3). The mixed solution was incubated for 30 min at 37° C. The reaction was stopped, and the cells were separated by cold centrifugation (4° C.) at 375×g

TABLE 1
Clinical data, results of serology, basophil histamine release and skin testing for the study population

| | | | | | | | | | Specific | | | | | | |
|---|---|---|---|---|---|---|---|---|---|---|---|---|---|---|---|
| | | | | | | | | | IgE | | | | | | |
| | | | | Positive | | | | IgE | (kU/L) | Total IgE | (S/T) | | | | |
| N | Initials | Age | Symptom | prick test | Food allergy | +ID test | 30% HR | class | (S) | (kU/L) (T) | % | IgG1 | IgG2 | IgG3 | IgG4 |
| 1 | F-T | 58 | R-C | m, b, o, g | a, c, p, al, n | 10⁻³ | 0.3 × 10⁻² | 3 | 12.1 | 30.6 | 39.5 | 0.24 | 0.15 | 0.181 | 0.131 |
| 2 | S-F | 33 | R-C | b | | 0.3 × 10⁻¹ | 0.3 × 10⁻² | 4 | 24.1 | 142 | 16.9 | 0.798 | 0.111 | 0.092 | 0.408 |
| 3 | W-F | 53 | R-C | b, o | a, p, n | 10⁻¹ | 10⁻¹ | 3 | 5.22 | 11.5 | 45.4 | 0.537 | 0.079 | 0.788 | 0.082 |
| 4 | F-JJ | 51 | R-C | b | | 10⁻³ | 0.3 × 10⁻³ | 5 | 59.9 | 128 | 46.8 | 1.066 | 0.106 | 0.072 | 0.198 |
| 5 | G-S | 49 | R-C | m, b | a, p, ap | 0.3 × 10 | 10⁻¹ | 3 | 17.1 | 33 | 51.8 | 0.187 | 0.074 | 0.065 | 0.075 |
| 6 | S-S | 50 | R-C | b, o, w, c | | 1 | 0.3 × 10⁻² | 4 | 41.1 | 168 | 24.5 | 0.287 | 0.081 | 0.081 | 0.103 |
| 7 | B-A | 39 | R-C, A | b, a | | 0.3 × 10 | 0.3 × 10⁻¹ | 4 | 20 | 43 | 46.5 | 0.298 | 0.083 | 0.081 | 0.071 |
| 8 | O-C | 37 | R-C | b, o, a, w | a, c, p | 10 | 10⁻¹ | 5 | 79.9 | 231 | 34.6 | 0.658 | 0.09 | 0.83 | 0.209 |
| 9 | L-N | 44 | R-C, A | b, a | a, n | 10⁻⁵ | 10⁻² | 4 | 26.5 | 115 | 23.1 | 1.325 | 0.098 | 0.074 | 0.136 |
| 10 | M-C | 43 | R-C, A | b, o | | 10⁻³ | 0.3 × 10⁻³ | 3 | 4.51 | 6.9 | 65.4 | 0.5 | 0.173 | 0.063 | 0.06 |
| 11 | H-C | 58 | R-C | b, o, a | a | 10⁻⁴ | 10⁻⁴ | 4 | 22.7 | 113 | 20 | 0.505 | 0.1 | 0.068 | 0.208 |
| 12 | P-D | 49 | R-C, A | m, b, a, g | a, c, ap, p, | 10⁻² | 1 | 3 | 17.4 | 94.5 | 18.4 | 1.043 | 0.075 | 0.065 | 0.129 |
| | | | | | n, ce, ca | | | | | | | | | | |
| 13 | H-M | 41 | R-C | m, b, o | a, c, p, al, n | 0.3 × 10⁻¹ | 10⁻² | 5 | 51.5 | 82.2 | 62.6 | 0.183 | 0.059 | 0.065 | 0.062 |
| 14 | W-S | 53 | R-C, A | m, b | a | 0.3 × 10⁻² | 0.3 × 10⁻¹ | 4 | 45.5 | 72.3 | 62.9 | 0.236 | 0.161 | 0.075 | 0.889 |
| 15 | B-E | 28 | R-C | b, g | n, al | 1 | 10⁻² | 3 | 14 | 90.9 | 15.4 | 0.389 | 0.069 | 0.079 | 0.078 |
| 16 | B-M | 50 | R-C | b, o, g | a, p | 0.3 × 10⁻² | 10⁻³ | 3 | 4.74 | 16.5 | 28.7 | 0.14 | 0.064 | 0.068 | 0.065 |
| 17 | W-B | 46 | R-C | m, b, g, | a, ca, k | 10⁻¹ | 10⁻¹ | 4 | 21.4 | 91.3 | 23.4 | 0.344 | 0.086 | 0.075 | 0.111 |
| | | | | o, w, c | | | | | | | | | | | |
| 18 | S-B | 40 | R-C | b | | 1 | 1 | 2 | 1.65 | NA | NA | 0.113 | 0.061 | 0.065 | 0.074 |

Symptoms: R, rhinitis; C, conjunctivitis; A, asthma.
Positive prick test: m, mites; b, birch; o, olive; g, grass; w, weeds; a, ash; c, cat.
Food allergy; a, apple; ap, apricot; c, cherry; p, peach; al, almond; n, nuts; k, kiwi; ce, celery, ca, carrot.
ID test, intradermal test;
HR, histamine release (values in μg/mL);
c.p.m., counts per minute.

for 5 min. 200 μl of the cell-free supernatant was used for histamine quantification in a radioimmunoassay with acylated histamine monoclonal antibodies (Immunotech, Marseille, France) as described previously (Morel A M, et al. J Allergy Clin Immunol (1988) 82:646-54). Total histamine was measured after cell lysis by repeated thawing and freezing. All experiments were performed in duplicate. The parameter used to describe the degree of basophil sensitivity was the lowest allergen concentration inducing 30% of total histamine release.

Intradermal Testing

Threshold intradermal skin tests were performed by injection of 0.03 ml of 10-fold dilutions of rBet v 1 on the lateral part of the arm. Serial dilutions were prepared from a solution of 1000 mg/ml and the first dilution tested was 10 mg/ml. The tests were read 15 min after injection. The area of weal and erythema was recorded. The test was considered positive when the induced weal area exceeded that of the weal induced by injection and the lowest concentration of allergen inducing a positive test result was used for comparison with other parameters (Grammer L C, et al., J Allergy Clin Immunol (1985) 76:123-7).

Statistical Analysis of the Data

Correlation between different parameters was tested by Spearman s non-parametric tests using VisualStats Professional software (version 2003).

Results

Poor Association Between rBet v 1-Specific Immunoglobulin E Levels and Skin Sensitivity to rBet v 1

To compare rBet v 1-specific IgE levels and skin sensitivity, rBet v 1-specific IgE levels were measured by CAP and correlated with the threshold concentration of rBet v 1 inducing a positive intradermal test weal reaction. FIG. 1 shows that there is no association between allergen-specific IgE levels and skin sensitivity (r=−0.007, P=0.977). In individual patients a strong discrepancy between allergen-specific IgE and skin sensitivity was observed. For example, patient 8 exhibited high Bet v 1-specific IgE level (79.9 kU/l) but showed a positive ID reaction only at 10 mg/ml of rBet v 1 (Table 1). On the other hand, patient 10 had low rBet v 1-specific IgE (4.5 kU/1), yet with a 1000-fold greater skin sensitivity to Bet v 1 (positive ID test reaction at 1 ng/ml of rBet v 1) than patient eight. Seven patients (2, 5, 7, 9, 11, 12 and 17) with similar rBet v 1-specific IgE levels (17.1 26.6 kU/l) exhibited an extremely broad range of skin sensitivity to rBet v 1 (from 3 to 10 5 mg/ml) (Table 1).

Poor Association Between rBet v 1-Specific Immunoglobulin E Levels and rBet v 1-Related Basophil Sensitivity

FIG. 2 (IgE vs. 30% histamine release) shows that there is also a lack of association between rBet v 1-specific IgE levels and Bet v 1-induced basophil sensitivity (FIG. 2: r=−0.113, P=0.656). The concentration of rBet v 1 required to induce 30% histamine release varied from 10⁻³ to 1 mg/ml. For given levels of rBet v 1-specific IgE (RAST class 3: 4.51 17.1 kU/l), the concentration of rBet v 1 inducing 30% histamine release varied 1000-fold (1 10⁻³ mg/ml).

Association Between rBet v 1-Induced Basophil Histamine Release and Skin Sensitivity

FIG. 3 shows that the results of intradermal testing and basophil histamine release tests are better associated than the results of serological and biological tests. There is a significant trend between the concentrations of rBet v 1 eliciting 30% histamine release and intradermal weal reactions (r=0.614; P=0.007). Patients with extremely bad association between rBet v 1-specific IgE levels and results of biological testing (e.g. patients 8 and 10) showed better association when intradermal testing results were compared with basophil histamine release (Table 1). Results of other tests performed in order to explain the discrepancies between serological and biological tests are given below.

Measurements of rBet v 1-Specific Immunoglobulin G Subclasses, Immunoglobulin A and Immunoglobulin M

It has been described that Bet v 1-allergic patients' sera contain Bet v 1-specific IgG antibodies that may interfere with IgE binding to Bet v 1 or recognise epitopes on the Bet v 1 molecule other than IgE and hence have no effect on IgE binding to Bet v 1 (Visco V, et al. J Immunol (1996) 157:956-62; Denepoux S, et al. FEBS Lett (2000) 465:39-46). Therefore the levels of rBet v 1-specific IgG were determined (IgG1 IgG4; Table 1). The patients exhibited varying rBet v 1-specific IgG1 IgG4 subclass responses with most pronounced responses in the IgG1 and IgG4 subclasses. No significant levels of rBet v 1-specific IgA and IgM antibodies were detected in the sera, excluding the possibility that these antibody classes may influence IgE recognition of Bet v 1.

Evaluation of Bet v 1-Specific Immunoglobulin E as a Percentage of Total Immunoglobulin E

If Bet v 1-specific IgE only accounts for a low percentage of total IgE, poor histamine release and skin reactivity might be explained by the fact that basophils and mast cells are primarily occupied by IgE directed against other allergens. Therefore the total IgE values were determined and the percentage of Bet v 1-specific IgE was calculated. The patients in this example had relatively low total IgE values (<168 kU/L) and no association between a low percentage of Bet v 1-specific IgE and poor biological responses was found. For example, in patient 11, who showed high sensitivity, Bet v 1-specific IgE only accounted for 20% of the total IgE. On the other hand, patient 13 was less sensitive, although 62.6% of the total IgE was directed against Bet v 1 (Table 1).

Discussion

The question of whether allergen-specific IgE antibody levels, effector cell sensitivities, and clinical sensitivity correlate remains a matter of controversy. Several studies have shown a significant correlation of allergen-specific serum IgE antibodies with allergen-induced immediate type reactions even when using a complex mixture of various allergenic and non-allergenic components, which may make it difficult to compare skin tests and RAST (Stenius B, et al., Clin Allergy (1971) 1:37-55; Bousquet J, et al., Clin Allergy (1987) 17:529-36; Norman P S, et al., J Allergy Clin Immunol (1973) 52:210-24; Lichtenstein L M, et al. J Allergy Clin Immunol (1971) 47:103 (A37)). Recently, other studies using purified allergens (Witteman A M, et al., J Allergy Clin Immunol (1996) 97:16-25) and recombinant allergens (Niederberger V, et al. J Invest Dermatol (2001) 117:848-51) have demonstrated considerable discrepancies between antibody levels and biological sensitivity.

A clinical study using a defined purified and structurally folded allergen (i.e. the major birch pollen allergen, Bet v 1) to investigate the relation between specific IgE, basophil degranulation, and skin sensitivity at a molecular level was performed. Good agreement between the three methodologies and clinical relevance of birch sensitivity was found; however, strong discrepancies were noted between the levels of allergen-specific IgE, the basophil sensitivity and in vivo sensitivity (i.e. skin sensitivity as determined by end-point titration). In certain patients, very low specific IgE levels but high sensitivity in basophil degranulation and skin tests and vice versa was observed. A review of the literature reveals the scarcity of studies comparing skin tests, basophil histamine release and specific IgE levels. The few available studies showed greatly varying results and were performed with crude allergen extracts. For example, Norman et al. (Norman P S, et al., J Allergy Clin Immunol (1973) 52:210-24) found that the three tests were in good agreement with each other in the diagnosis of ragweed hayfever. Lichtenstein et al. (Lichtenstein L M, et al. J Allergy Clin Immunol (1971) 47:103 (A37)) found a quantitatively significant relationship between skin tests and histamine release. However, no measurement of specific IgE was performed in this example. The response of sensitised leucocytes and mast cells to antigen can depend on a great variety of factors.

One possibility for low sensitivity and poor release of histamine would be that only a small proportion of the total serum IgE accounts for allergen-specific IgE. Therefore the total IgE levels were determined and the percentage of allergen-specific IgE was calculated. However, an association between low percentages of allergen-specific IgE responses and poor biological activity was found. The possibility that a low percentage of specific IgE out of the total IgE may be responsible for poor biological responses towards the given allergen may be of greater importance in polysensitised subjects (Norman P S, et al., J Allergy Clin Immunol (1973) 52:210-24; Conroy M C, et al. J Immunol (1977) 118:1317-21; MacGlashan D W Jr, et al., J Immunol (1986) 136:2231-9).

There are several other factors that may be responsible for the discrepancy between allergen-specific IgE levels and biological responses but they cannot be addressed even in a system using purified allergens. They include interindividual differences in basophil and mast cell sensitivities because of variability in IgE-receptor cell surface density, a parameter that is regulated by serum IgE levels (Conroy M C, et al. J Immunol (1977) 118:1317-21; Malveaux F J, et al., J Clin Invest (1978) 62:176-81; Dembo M, et al., J Immunol (1978) 121:345-53; MacGlashan D W Jr, et al. J Allergy Clin Immunol (1999) 104:492-8). Different cell sensitivities have been demonstrated by variable shifts of the dose response curves (measured by 50% or 30% sensitivity) in case of similar total and antigen-specific IgE serum concentrations (Conroy M C, et al. J Immunol (1977) 118:1317-21; MacGlashan D W Jr., J Allergy Clin Immunol (1993) 91:605-15).

Furthermore, it has been shown that persons with equivalent numbers of IgE molecules on basophils may release 0-100% of their histamine content (Conroy M C, et al. J Immunol (1977) 118:1317-21). The same has been observed for cutaneous mast cells (Petersen L J, et al., J Allergy Clin Immunol (1996) 97:672-9; Bordignon V, Pet al., Invest Allergol Clin Immunol (2000) 10:78-82). In addition, it has been shown that early signal events occur involving sykkinase and IP3 products, which are not linked to the level of specific IgE or basophil sensitivity (MacGlashan D W Jr., J Allergy Clin Immunol (1993) 91:605-15; Miura K, et al., J Immunol (2001) 167:7027; MacGlashan D W Jr., J Immunol (2003) 170:4914-25).

Recent evidence indicates that mast cells may also be influenced via Toll-like receptors (Marshall J S, et al., Int Arch Allergy Immunol (2003) 132:87-97). However, the rBet v 1 preparation used for the experiments did not contain endotoxins.

Finally, it is possible that the presence of IgE antibodies with varying affinities or binding specificities for epitopes inducing varying anaphylactic activity may have influenced serological and biological test results.

In conclusion, this study demonstrates on a molecular level that allergen-specific serum IgE levels are not necessarily related to the biological sensitivity as determined by cellular and in vivo tests. A moderate association was, however, found between the cutaneous tests and the basophil histamine release tests.

### Example 2

To determine the sensitivity of a patient before therapy to allow the choice of the correct dose a whole blood basophil histamine release test is used. Patients with high sensitivity will be injected smaller doses than less sensitive patients. Before treatment a dose response curve will be established with purified allergen. In parallel, cells will be stimulated with anti-IgE to determine overall cell sensitivity which may affect sensitivity to the allergen. Success of treatment should be controlled after IgG antibodies against the allergen become detectable which is usually the case after 4-8 weeks of treatment. Since blocking of IgG antibodies may be responsible for the reduction of sensitivity it may be useful to determine in parallel IgG levels to the given allergen. Again a dose response is determined with the purified allergen and anti-IgE. Either the dose giving maximal cell activation (i.e., maximal histamine release or CD203c upregulation) is compared or the dose giving a certain degree of activation is determined and compared with the test result obtained before treatment. Materials and methods are as described in example 1.

### Example 3

When basophil histamine release experiments were performed with washed granulocyte preparations as described (Stahl-Skov et al. 1977. J Exp Immunol 27: 432-439) no correlation between histamine release data and skin sensitivity was found.

Histamine release was done using basophils from allergic patients. They were enriched by Dextran sedimentation, isolated, washed, re-suspended in histamine release buffer, and exposed to different concentrations of recombinant Bet v 1 (10⁻⁵, 10⁻⁴, 10⁻³, 10⁻², 10⁻¹, 1 μg/ml) or anti-IgE mAb E-124-2-8 (1 μg/ml) in 96-well microtiter plates (TPP, Trasadingen, Switzerland) for 30 minutes at 37° C. After incubation, cells were centrifuged. Cell-free supernatants were recovered and analyzed for histamine content by using a commercial radioimmunoassay (Immunotech, Marseille, France). Histamine release was expressed as a percentage of total histamine measured in cell lysates (Valent et al., 1989, Proc Natl Acad Sci USA 86: 5542-5546).

Skin prick tests were performed with serial dilutions (1:2) of recombinant Bet v 1 as described (Pauli et al., 1996, J Allergy Clin Immunol 97: 1100-1109).

Maximal histamine released from basophils exposed to recombinant Bet v 1 (HR %-max) did not correlate with skin prick test reactions (mm²) (SPT 2 μg/ml) (r=0.224, P=0.342) (FIG. 5).

1. Method for evaluating the allergen sensitivity of an individual and/or the clinical efficacy of an allergen immunotherapy comprising the steps:
providing at least two samples selected from the group consisting of blood or fractions thereof, connective tissue, nasal, bronchial, skin or gut biopsy material from an individual subjected or intended to be subjected to an immunotherapy with at least one pure allergen or derivative thereof, wherein the samples contain cells capable of releasing mediators in response to said allergen;
contacting said sample with said allergen or derivative thereof, and
determining the amounts of mediators released from said sample and
evaluating the allergen sensitivity of the individual prior to therapy and/or the clinical efficacy of the immunotherapy by comparing said amounts.

2. (canceled)

3. The method according to claim 1 characterized in that the mediators are selected from the group consisting of histamine, tryptase, prostaglandins, leukotrienes, especially cysteinyl leukotrienes, eosinophil cationic protein, cytokines, like interleukins (IL), IL-2R, CD63, CD203c and combinations thereof.

4. The method according to claim 1 characterized in that said cells are mast and/or basophilic and/or eosinophilic cells.

5. The method according to claim 1 characterized in that the sample further comprises immunoglobulins (Ig).

6. The method according to claim 1 characterized in that the samples are provided before and after subjecting said individual to an immunotherapy.

7. The method according to claim 1 characterized in that the samples are provided after subjecting said individual to an immunotherapy.

8. The method according to claim 1 characterized in that the at least one sample is provided after a maximum of about 1 hour, about 12 hours, about 24 hours, about 10 days, about 4 weeks, about 6 months and about 36 months, after subjecting said individual to an immunotherapy.

9. The method according to claim 1 characterized in that said allergen is recombinantly produced.

10. The method according to claim 9, characterized in that said allergen comprises at least one deletion, at least one substitution or at least one insertion.

11. The method according to claim 9, characterized in that said allergen is modified by reshuffling the fragments of said allergen by genetic engineering.

12. The method according to claim 1 characterized in that said sample is contacted with varying concentrations of said allergen.

13. The method according to claim 12, characterized in that the concentration of said allergen is selected within the range of about 1 ng/ml to about 100 μg/ml.

14. The method according to claim 1 characterized in that further total amount of the mediator of said cells is determined.

15. The method according to claim 14, characterized in that a degree of cellular sensitisation is defined by determining the concentration of said allergen inducing the release of about 10%, preferably about 30%, of the total amount of the mediator of said cells.

16. The method according to claim 15, characterized in that the allergen sensitivity of an individual and/or the clinical efficiency of an allergen immunotherapy is evaluated by observing the degree of cellular sensitisation in the course of said immunotherapy.

17. The method according to claim 1 characterized in that the mediator in the sample is determined by an immunological method, a chromatographical method, or both.

18. The method according to claim 17 characterised in that the method is selected from the group consisting of radioimmunoassay (RIA), enzyme linked immunosorbent assay (ELISA), high performance liquid chromatography (HPLC), reverse transcriptase polymerase chain reaction, immunofluorescence flow cytometry and combinations thereof.

19. The method according to claim 1 characterized in that said allergen is selected from the group of the major birch pollen allergens, Bet v 1 and Bet v 4, the major timothy grass pollen allergens, Phl p 1, Phl p 2, Phl p 5, Phl p 6 and Phl p 7, the major house dust mite allergens, Der p 1 and Der p 2, the major cat allergen Fel d 1, the major bee allergens, the major wasp allergens, profilins, Phl p 12, and storage mite allergens, Lep d 2.

20. Kit for evaluating the allergen sensitivity of an individual or the clinical efficiency of an allergen immunotherapy for at least one allergy comprising
at least one allergen for inducing a mediator release of cells capable of releasing mediators in response to an allergen,
means for detecting the mediator, and
optionally at least one mediator standard.

21. (canceled)

22. The kit according to claim 20 characterized in that said cells are mast and/or basophilic and/or eosinophilic cells.

23. The kit according to claim 20 characterized in that said allergen is selected from the group consisting of major birch pollen allergens, Bet v 1 and Bet v 4, major timothy grass pollen allergens, Phl p 1, Phl p 2, Phl p 5, Phl p 6 and Phl p I 1 major house dust mite allergens, Der p 1 and Der p 2, major cat allergen Fel d 1, major bee allergens, major wasp allergens, profilins, Phl p 12, and storage mite allergens, Lep d 2.

24. The kit according to claim 20 characterized in that the means for detecting the mediator are selected from the group consisting of antibodies.

25. The method according to claim 5 characterized in that the sample further comprises immunoglobulin G (IgG).

26. The method according to claim 13, characterized in that the concentration of said allergen is selected within the range of about 1 pg/ml to about 10 μg/ml.

27. A kit for evaluating the allergen sensitivity of an individual or the clinical efficiency of an allergen immunotherapy for at least one allergy comprising:
at least two of the following components
at least one allergen for inducing a mediator release of cells capable of releasing mediators in response to an allergen,
means for detecting the mediator,
at least one mediator standard, and
cells capable of releasing mediators in response to an IgE-allergen complex.

28. The kit according to claim 27, characterized in that said cells are mast and/or basophilic and/or eosinophilic cells.

29. The kit according to claim 27 characterized in that said allergen is selected from the group consisting of major birch pollen allergens, Bet v 1, Bet v 4, major timothy grass pollen allergens, Phl p 1, Phl p 2, Phl p 5, Phl p 6, Phl p I 1 major house dust mite allergens, Der p 1, Der p 2, major cat allergen Fel d 1, major bee allergens, major wasp allergens, profilins, Phl p 12, storage mite allergens, Lep d 2 and combinations thereof.

30. The kit according to claim 27 characterized in that the means for detecting the mediator are selected from the group consisting of antibodies.

31. Method for evaluating the allergen sensitivity of an individual and/or the clinical efficacy of an allergen immunotherapy comprising the steps:
providing cells capable of releasing mediators in response to an IgE-allergen complex,
contacting said cells with serum and/or plasma of said individual spiked with at least one pure allergen or derivative thereof, and
determining the amounts of mediators released from said sample and evaluating the allergen sensitivity of the individual prior to therapy and/or the clinical efficacy of the immunotherapy by comparing said amounts.

32. The method according to claim 31 characterized in that the mediators are selected from the group consisting of histamine, tryptase, prostaglandins, leukotrienes, cysteinyl leukotrienes, eosinophil cationic protein, cytokines, interleukins (IL), IL-2R, CD63, CD203c and combinations thereof.

33. The method according to claim 31 characterized in that said cells are mast and/or basophilic and/or eosinophilic cells.

34. The method according to claim 31 characterized in that the sample further comprises immunoglobulins (Ig).

35. The method according to claim 31 characterized in that the samples are provided before and after subjecting said individual to an immunotherapy.

36. The method according to claim 31 characterized in that the samples are provided after subjecting said individual to an immunotherapy.

37. The method according to claim 31 characterized in that the at least one sample is provided after a maximum of about 1 hour, about 12 hours, about 24 hours, about 10 days, about 4 weeks, about 6 months and about 36 months, after subjecting said individual to an immunotherapy.

38. The method according to claim 31 characterized in that said allergen is recombinantly produced.

39. The method according to claim 31, characterized in that said allergen comprises at least one deletion, at least one substitution or at least one insertion.

40. The method according to claim 31, characterized in that said allergen is modified by reshuffling the fragments of said allergen by genetic engineering.

41. The method according to claim 31, characterized in that said sample is contacted with varying concentrations of said allergen.

42. The method according to claim 31, characterized in that the concentration of said allergen is selected within the range of about 1 ng/ml to about 100 μg/ml.

43. The method according to claim 31, characterized in that the concentration of said allergen is selected within the range of about 1 pg/ml to about 10 μg/ml.

44. The method according to claim 31, characterized in that further total amount of the mediator of said cells is determined.

45. The method according to claim 31, characterized in that a degree of cellular sensitisation is defined by determining the concentration of said allergen inducing the release of about 10% of the total amount of the mediator of said cells.

46. The method according to claim 31, characterized in that a degree of cellular sensitisation is defined by determining the concentration of said allergen inducing the release of about 30% of the total amount of the mediator of said cells.

47. The method according to claim 31, characterized in that the allergen sensitivity of an individual and/or the clinical efficiency of an allergen immunotherapy is evaluated by observing the degree of cellular sensitisation in the course of said immunotherapy.

48. The method according to claim 31, characterized in that the mediator in the sample is determined by an immunological method, a chromatographical method or both methods.

49. The method according to claim 48 characterized in that the method is selected from the group consisting of radioimmunoassay (RIA), enzyme linked immunosorbent assay (ELISA), high performance liquid chromatography (HPLC), reverse transcriptase polymerase chain reaction, immunofluorescence flow cytometry and combinations thereof.

50. The method according to claim 31, characterized in that said allergen is selected from the group of the major birch pollen allergens, Bet v 1, Bet v 4, the major timothy grass pollen allergens, Phl p 1, Phl p 2, Phl p 5, Phl p 6 Phl p 7, the major house dust mite allergens, Der p 1 and Der p 2, the major cat allergen Fel d 1, the major bee allergens, the major wasp allergens, profilins, Phl p 12, and storage mite allergens, Lep d 2.
